(19) United States
(12) Patent Application Publication
(10) Pub. No.: US 2025/0177448 A1
(43) Pub. Date: Jun. 5, 2025

(54) NEW USE OF PEG-PHOSPHOLIPID MOLECULES

(71) Applicant: ICOAT MEDICAL AB, Stockholm (SE)

(72) Inventors: Bo NILSSON, Uppsala (SE); Yuji TERAMURA, Tokyo (JP); Felix SELLBERG, Uppsala (SE)

(21) Appl. No.: 18/840,203

(22) PCT Filed: Mar. 7, 2023
(86) PCT No.: PCT/SE2023/050205

§ 371 Date: Aug. 21, 2024

(30) Foreign Application Priority Data
Mar. 7, 2022 (SE) 2250304-9
Aug. 12, 2022 (SE) 2250959-0

(51) Int. Cl. A61K 35/18; A61K 47/24; A61K 47/60; G01N 33/569

(52) U.S. Cl. CPC A61K 35/18; A61K 47/24; A61K 47/60; G01N 33/56977; G01N 2333/70539

(57) ABSTRACT

The present invention relates to the use of PEG-phospholipid molecules to selectively mask surface antigens on erythrocytes, thrombocytes and/or endothelial cells. Blood products treated with PEG-phospholipid molecules can thereby be infused into uncrossmatched or incompatible recipients with reduced risk of antibody binding to the surface antigens and agglutination. Correspondingly, organ transplants treated with PEG-phospholipid molecules can be transplanted into uncrossmatched or incompatible recipients with reduced risk of antibody binding and antibody-mediated rejection.

### TECHNICAL FIELD

The present invention generally relates to poly(ethylene glycol)phospholipid (PEG-phospholipid) molecules and to uses thereof in selective masking of cell surface antigens.

### BACKGROUND

A donor-recipient test, also called cross-matching or crossmatching, is a test performed before a blood transfusion as part of blood compatibility testing. Normally, this involves adding the recipient's blood plasma to a sample of the donor's red blood cells. If the donor's blood is incompatible with the recipient, antibodies in the recipient's blood will bind to antigens on the donor red blood cells. This antibody-antigen reaction causes clumping, so called agglutination, or destruction of the red blood cells.

Along with blood typing of the donor and recipient and screening for unexpected blood group antibodies, cross-matching is one of a series of steps in pre-transfusion testing. A complete cross-matching process takes up to 1 hour and is thereby not always used in emergencies. In such emergent situations, O Rh negative blood may be given to the patients, to which no preformed antibodies exist. This type of blood is, however, rare and cannot be given in large quantities.

ABO cross-matching and complement dependent cytotoxicity (CDC) assay are also used to determine compatibility between a donor and recipient in organ transplantation where pre-existing anti-ABO or anti-class I human leukocyte antigen (HLA) antibodies may exist. If the recipient has received multiple transfusions or has given birth to a child, there is a risk for immunization against one or several of the more than 50 blood group antigens. Such pre-existing antibodies, called irregular antibodies, make the selection of compatible blood difficult. Furthermore, pre-existing anti-ABO and anti-class I HLA antibodies may also cause reduced circulation time for transfused platelets administered to patient to prevent bleeding due to thrombocytopenia.

WO 2004/050897 discloses methods for the preparation of an RBC composition having reduced antigenicity and having reduced levels of hemolysis. The methods involve reaction of an activated antigen masking compound having a molecular weight of approximately 20-40 kDa, wherein the resulting red cells are not readily hemolyzed by any serum or plasma sample, for example by complement lysis. The RBC compositions are of particular use for introduction into an individual in cases where the potential for an immune reaction is high, for example in alloimmunized blood recipients or in trauma situations where the possibility of transfusion of a mismatched unit of blood is higher.

Advanced Drug Delivery Reviews 62:827-840 (2010) provides a review of various attempts of encapsulating islets of Langerhans or producing bioartificial pancreases to isolate the islets from the recipient's immune system.

There is, still, a need for a treatment of blood products that can be used, for instance, in situations of emergency when a complete cross-matching process between donor and recipient is not possible. There is also a need for a treatment of transplants in order to treat antibody-mediated rejection in connection with transplantation.

### SUMMARY

It is a general objective to inhibit antibody binding and agglutination in connection with blood transfusion.

It is another general objective to inhibit antibody binding and antibody-mediated rejection in connection with organ transplantation.

These and other objectives are met by the invention as defined herein.

The invention is defined in the independent claims. Further embodiments of the invention are defined in the dependent claims.

An aspect of the invention relates to poly(ethylene glycol) phospholipid (PEG-phospholipid) molecules for use in selective masking surface antigens of erythrocytes and/or thrombocytes in a blood product from a donor to inhibit antibody binding to the surface antigens in connection with transfusion of the blood product into an uncrossmatched or incompatible recipient. A PEG chain of the PEG-phospholipid molecules has an average molecular weight selected within the range of from 3 000 up to 10 000 Da.

Another aspect of the invention relates to PEG-phospholipid molecules for use in selective masking of surface antigens of an organ transplant comprising erythrocytes, thrombocytes and/or endothelial cells from a donor to inhibit antibody binding to the surface antigens and antibody-mediated rejection (AMR) in connection with transplantation of the organ transplant into an uncrossmatched or incompatible recipient.

A further aspect of the invention relates to a blood product comprising erythrocytes and/or thrombocytes and PEG-phospholipid molecules anchored in the cell membrane of the erythrocytes and/or thrombocytes and masking surface antigens of the erythrocytes and/or thrombocytes. A PEG chain of the PEG-phospholipid molecules has an average molecular weight selected within the range of from 3 000 up to 10 000 Da.

Yet another aspect of the invention relates to an in vitro method of treating erythrocytes and/or thrombocytes comprising selective masking surface antigens of erythrocytes and/or thrombocytes by adding in vitro PEG-phospholipid molecules to the erythrocytes and/or thrombocytes. A PEG chain of the PEG-phospholipid molecules has an average molecular weight selected within the range of from 3 000 up to 10 000 Da.

Another aspect of the invention relates to a blood transfusion method comprising adding PEG-phospholipid molecules to a blood product from a donor to selectively mask surface antigens on erythrocytes and/or thrombocytes present in the blood product. A PEG chain of the PEG-phospholipid molecules has an average molecular weight selected within the range of from 3 000 up to 10 000 Da. The method also comprises transfusing the blood product into an uncrossmatched or incompatible recipient.

A further aspect of the invention relates to an irregular antibody screening method. The method comprises adding PEG-phospholipid molecules to a blood sample from a subject to selectively mask surface antigens on erythrocytes present in the blood sample. A PEG chain of the PEG-phospholipid molecules has an average molecular weight selected within the range of from 3 000 up to 10 000 Da. The method also comprises screening for irregular antibodies bound to erythrocytes present in or obtained from the blood sample.

PEG-phospholipid molecules of the embodiments can selectively mask surface antigens on erythrocytes, thrombocytes and/or endothelial cells. Blood products treated with the PEG-phospholipid molecules can thereby be infused into uncrossmatched or incompatible recipients with reduced risk of antibody binding to the surface antigens and agglutination. Correspondingly, organ transplants treated with PEG-phospholipid molecules can be transplanted into uncrossmatched or incompatible recipients with reduced risk of antibody binding and AMR.

### BRIEF DESCRIPTION OF THE DRAWINGS

The embodiments, together with further objects and advantages thereof, may best be understood by making reference to the following description taken together with the accompanying drawings, in which:

FIG. 1. Illustration of the mechanism of hydrophobic interaction of the PEG-phospholipid molecule to the lipid bilayers of the cell membrane.

FIG. 2. Flow cytometric analysis of FITC-labelled PEG-phospholipid binding to CCRF-CEM cells.

FIG. 3. In vitro results describing the mechanism of action (MOA) of PEG-phospholipid. (A) Inhibition of binding to surface antigens when cells are coated with PEG-phospholipid. (B) Inhibition of binding to blood group antigens when RBCs are coated with PEG-phospholipid. (C) Inhibition of complement mediated lysis of RBCs.

FIG. 4. Binding to surface antigens using antibodies directed against large and small surface antigens (CD52 and CD4, respectively). Coating of cells with PEG-phospholipid results in a near complete inhibition of binding to a small surface antigen.

FIG. 5. Agglutination test cards for Rh phenotyping. (A) 0 Rh+ blood sample treated with PEG-phospholipid molecules. (B) 0 Rh+ control blood sample.

FIG. 6. Agglutination test cards for Rh phenotyping. (A) A Rh+ blood sample treated with PEG-phospholipid molecules. (B) A Rh+ control blood sample.

FIG. 7. Agglutination test cards for Rh phenotyping. (A) A Rh+ blood sample treated with PEG-phospholipid molecules. (B) A Rh+ control blood sample.

FIG. 8. Binding of the LP recognition molecule CL-11 (n=5) on native and coated HUVEC after exposure to human plasma, and of concanavalin A (a lectin of broad specificity) without exposure to plasma (n=3) was analyzed by flow cytometry. The values for CL-11 binding are presented after subtraction of the binding of CL-11 in the presence of 10 mM EDTA. It was shown that the PEG-phospholipid coating was able to mask the acceptor molecules for both concanavalin A and (to a lesser extent) CL-11. Statistical evaluation by one-sample t-test and one-way ANOVA.

FIG. 9. Binding of the LP recognition molecule MBL on native and coated HUVEC after exposure to human plasma was analyzed by flow cytometry.

FIG. 10. Detachment of PEG-phospholipid from cells was analyzed by incubating three million CCRF-CEM cells with 50 μL of FAM-PEG-phospholipid (2 mg/mL) for 30 min at room temperature (n=3). After washing, the cells were cultivated for 7 days, and the bound amount of PEG-phospholipid was assessed by flow cytometry (left panel) and confocal microscopy (right panel).

FIG. 11. Two donor pigs underwent bilateral nephrectomy. The kidneys were then procured and kept in cold storage for 24 h. Biotin-labeled PEG-phospholipid was administered and incubated in both kidneys for 40 min. After 40 min, non-bound biotin-PEG-phospholipid was flushed from the system and the kidneys were transplanted to recipient pigs, and initial biopsies were taken from the treated and native kidneys at transplantation and after 60 min (n=4). Pigs were euthanized sequentially after 12, 24, 48, and 72 h, and biopsies were taken from the transplanted and native kidneys. Fluorescence was detected via secondary FITC-streptavidin and confocal microscopy. Fluorescence in native kidneys was compared with transplanted kidneys. The half-life of PEG-phospholipid was calculated from fluorescence images, a one-phase exponential decay model was applied to fit a curve to the data points and t½ was calculated to be 14 hrs (95% confidence interval 8-26).

FIG. 12. Detection of fluorescence in the urine of 4 of the PEG-phospholipid treated and native kidneys in the ANSM, corroborating that the PEG-phospholipid is excreted by the kidneys. Two-way repeated measure ANOVA (coated/uncoated); p=0.0013.

FIG. 13. Wedge biopsies were taken at 1 min, 60 min and 360 min and quantified immunohisto-chemically by confocal microscopy for protein expression of nitrotyrosine, HO-1 and INOS. No significant difference was found between treated and non-treated tissue. Two-way repeated measure ANOVA (coated/uncoated); ns.

FIG. 14. Complement (C3a, sC5b-9), coagulation (TAT) and contact system (FXIIa-C1INH) markers were serially sampled from the kidney vein of the PEG-phospholipid-treated kidneys and their controls the first 60 min; thereafter systemically until 360 min. Two-way repeated measure ANOVA (coated/uncoated); TAT p=0.0042; C3a p=0.0002; sC5b-9 p=0.0042; FXIla/C1INH p=0.0029).

FIG. 15. Wedge biopsies were taken at 1 min, 60 min and 360 min and analyzed immunohisto-chemically for C4d, and C3b. Two-way repeated measure ANOVA (coated/uncoated); C4d ns; C3b p=0.013.

FIG. 16. Complement (C3a, sC5b-9), and coagulation (TAT) markers as indicators of thromboinflammation were serially sampled from the kidney vein of the PEG-phospholipid-treated kidneys and their controls at 60 min; thereafter systemically at 24, 48, 72 and 96 hours. Two-way repeated measure ANOVA (coated/uncoated); TAT p=0.006; C3a p=0.0059; sC5b-9 p=0.0023) (n=6 treated+4 controls).

FIG. 17. Wedge biopsies were taken at 5 min, and after 96 hours and analyzed immunohistochemically for C3b and MAC. Two-way repeated measure ANOVA (coated/uncoated); C3b ns; sC5b-9 ns (n=6 treated+4 controls).

FIG. 18. Analyzes of NETs was performed in biopsies from the ANSM by electron microscopic imaging and showed a strong deposition of NETs in the non-treated controls. In the untreated kidney (upper row), dense web-like structures (NETosis) were already observed in the 1-minute post-reperfusion biopsies and was mainly located within the Bowman's space (BS), tubules and peritubular capillaries (PTC). In the PEG-phospholipid treated kidneys (lower panels), NETs formation was markedly reduced and absent in larger area of the kidney tissue. Resting neutrophilic granulocytes were visible in the PEG-phospholipid treated kidneys (representative images of 6 analyzed).

FIG. 19. The PEG-phospholipid-treated kidneys exhibited a highly significant reduction in the deposition of NETs in all locations (n=6). Two-way repeated measure ANOVA (coated/uncoated); glomerular p≤0.0001; tubular p=0.0010; vascular p=0.0011.

FIG. 20. Cytokine mRNA expression of proinflammatory cytokines in the ANSM: RT-PCR of proinflammatory cytokines and TF in wedge biopsies from the individual kidneys and shown to be suppressed during the whole observation period. Expressed as percentage of the individual values at reperfusion (5 min) Two-way repeated measure ANOVA (coated/uncoated); IL-1 p=0.0021; IL-6 p<0.0001; TNF p=0.0077; TF p=0.0019) (n=6).

FIG. 21. Protein levels of proinflammatory cytokines in the ANSM: Serial samples from the kidney vein of the PEG-phospholipid-treated kidneys and their controls were drawn the first 60 min and thereafter systemically until 360 min. Two-way repeated measure ANOVA (coated/uncoated); IL-1B ns; IL-6 p=0.0005; TNF p=0.0328; TF p=0.0103) (n=6).

FIG. 22. Protein levels of cytokines in the SM: Cytokines were serially sampled from the kidney vein of the PEG-phospholipid-treated kidneys and their controls at 5 min and 60 min; thereafter systemically after 96 hours. IL-1a, and IL-12 were suppressed by PEG-phospholipid treatment already at 5 min in most cases. At 60 min all cytokines were stabilized at a low steady state representing a control value. After 96 hours all cytokines were still suppressed to baseline levels in the treated animals except for IL-1RA and IL-8, which were unaffected. Two-way repeated measure ANOVA (coated/uncoated); IL-1α, IL-β, INFγ, IL-2, IL-4, IL-6, IL-10, IL-12, and IL-18 p<0.01; IL-1RA, TNF and IL-8 ns (n=6 treated+4 controls).

FIG. 23. Kidney function monitored by plasma creatinine levels before and after transplantation in the SM (including CLSM and the in vivo detachment study). Plasma creatinine was estimated at baseline pre-transplantation and then daily at 24, 48, and 72, 96 hours. Two-way repeated measure ANOVA (coated/uncoated); p=0.0179 (n=6 treated+4 controls).

FIG. 24. The corresponding data in the CLSM. Two-way repeated measure ANOVA (coated/uncoated); p=0.0252 (n=5 treated+5 controls).

FIG. 25. PEG-phospholipids inhibited anti-HLA class I antibodies W6/32 from binding to PBMCs.

FIG. 26. PEG-phospholipids inhibited anti-HLA-ABC G46-2.6 from binding to PBMCs.

### DETAILED DESCRIPTION

The present invention generally relates to poly(ethylene glycol) phospholipid (PEG-phospholipid) molecules and to uses thereof in selective masking of cell surface antigens.

Traditionally, cross-matching is performed for blood products used in blood transfusion and for organs to be transplanted from a donor to a recipient. Such cross-matching is used to prevent agglutination in connection with blood transfusion or antibody-mediated rejection (AMR) of the transplanted organ. Both of these processes are mediated by antibodies of the recipient against surface antigens present on cells in the donor's blood or organ. However, in emergency situations, there may not be sufficient time available to perform a complete cross-matching process to save the life of the recipient. There is therefore a need for a treatment of blood products and/or organs that can be used to suppress the deleterious reactions, such as agglutination or AMR, otherwise occurring when infusing uncrossmatched blood or transplanting an uncrossmatched organ.

The present invention relates to the use of poly(ethylene glycol) phospholipids (PEG-phospholipids) molecules in creating a transient, non-toxic, artificial layer on the surface of cells in a blood product or organ. This shielding PEG-layer effectively masks surface antigens on the surface of the cells from a recipient's antibodies against the surface antigens. As a consequence, the PEG-phospholipid treatment of the blood product or of the organ prior to infusion or transplantation reduces the risk of antibody binding to the surface antigens, and thereby reduces the risk of agglutination or AMR by masking surface antigens from antibodies in the recipient body.

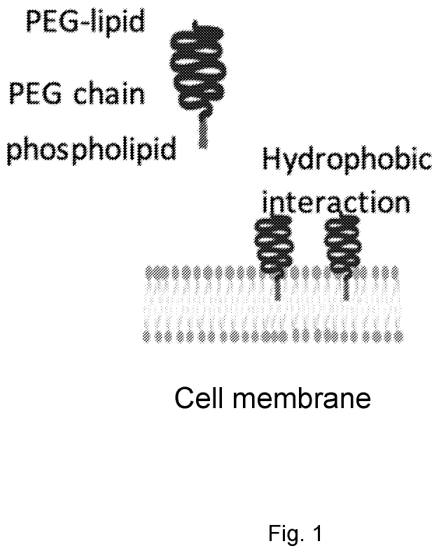

The PEG-phospholipid molecule has, with its phospholipid domain, the ability to uniformly anchor to the lipid membrane of cells by hydrophobic interactions creating a non-static artificial layer, see FIG. 1. The PEG domain of the PEG-phospholipid molecule is negatively charged and has the ability to shield surfaces from close interactions with macromolecules, including antibodies. Furthermore, cell surface polymer coating (CSPC) with the PEG-phospholipid molecule prevents non-specific adsorption of macromolecules, such as albumin. This is an important finding since intracellular adsorption of proteins is known to induce biological responses, such as activation of the coagulation and the complement system.

Experimental data as presented herein show that PEG-phospholipid molecules of the embodiments are able to mask blood group antigens, such as A and B antigens, and Rhesus factors, such as RhD, RhC, Rhc, RhE and Rhe, present on endothelial cells, i.e., red blood cells. As a consequence of this surface antigen masking, agglutination and cell lysis were inhibited by masking the surface antigens from antibodies and thereby preventing, or at least significantly inhibiting, binding of antibodies to the surface antigens on the erythrocytes.

Furthermore, experimental data as presented herein shows that the masking of surface antigens as achieved by the PEG-phospholipid molecules of the embodiments is selective in terms of having a masking effect that is dependent on the length of the PEG-polymer versus size of the surface antigen on the cell surface. For instance, a PEG-phospholipid molecule having a PEG domain or chain with a molecular weight of about 5 kDa has a length of approximately 6 nm leading to a 6 nm PEG layer when anchored in the cell membrane. Such a PEG-phospholipid molecule can thereby selectively mask surface antigens extending a distance from the cell surface of no more than about 6 nm or slightly above 6 nm. For instance, such a PEG-phospholipid molecule was capable of effective masking of blood group B antigen and cluster of differentiation (CD52), also referred to as CAMPATH-1 antigen, both of which have a size from the cell surface of about 1 nm. Furthermore, a partial masking was obtained of CD8, whereas no significant masking was seen for CD4. The latter extends about 15 nm from the cell surface, whereas the stalk like region of CD8 makes the molecule less protrusive from the cell membrane even though CD8 and CD4 are of similar size.

An aspect of the invention relates to PEG-phospholipid molecules for use in selective masking surface antigens of erythrocytes and/or thrombocytes in a blood product from a donor to inhibit antibody binding to the surface antigens in connection with transfusion of the blood product into an uncrossmatched or incompatible recipient. A PEG chain of the PEG-phospholipid molecules has an average molecular weight selected within the range of from 3 000 up to 10 000 Da.

A related aspect of the invention defines use of PEG-phospholipid molecules for the manufacture of a medicament for inhibiting antibody binding to surface antigens in connection with transfusion of a blood product comprising erythrocytes and/or thrombocytes having the surface antigens selectively masked by the PEG-phospholipid molecules into an uncrossmatched or incompatible recipient. A PEG chain of the PEG-phospholipid molecules has an average molecular weight selected within the range of from 3 000 up to 10 000 Da.

The selective masking of the surface antigens of erythrocytes and/or thrombocytes by the PEG-phospholipid molecules of the embodiments thereby inhibit agglutination by inhibiting antibody binding to the surface antigens in connection with transfusion of the blood product into an uncrossmatched or incompatible recipient. Hence, this aspect of the invention relates to PEG-phospholipid molecules for use in selective masking surface antigens of erythrocytes and/or thrombocytes in a blood product from a donor to inhibit agglutination in connection with transfusion of the blood product into an uncrossmatched or incompatible recipient.

It was highly surprising that PEG-phospholipid molecules having a PEG chain or domain with an average molecular weight selected within the range of from 3 up to 10 kDa could be used be used to selectively mask surface antigens of erythrocytes and/or thrombocytes given the disclosure in WO 2004/050897. This document reacts activated PEG compounds having a molecular weight of 20-40 kDa to covalently attach the PEG compounds on the surface of red blood cells. Experimental data presented in this document indicated that methoxy-PEG (mPEG) needed to have a molecular weigh to at least 20 kDa to efficiently mask antigens on the red blood cells and thereby inhibit antibody binding to these cells. When smaller mPEG molecules (5 kDa and 10 kDa) were tested it was concluded that they caused lysis of the red blood cells at the levels required to mask the antigens.

The PEG-phospholipid molecules of the present invention having smaller PEG chains (3-10 kDa) as compared to WO 2004/050897 (20-40 kDa) did not cause any cell lysis at the levels that were able to efficiently mask surface antigens on red blood cells. Hence, the usage of PEG-phospholipid molecules having phospholipid domains to anchor the PEG-phospholipid molecules in the cell membrane provides significant advantages as compared to chemically reacting mPEG molecules with proteins and other macromolecules anchored in the cell membrane of red blood cells.

The blood product is typically a whole blood product but could alternatively be a treated and/or processed blood product comprising erythrocytes (red blood cells), thrombocytes (platelets) or erythrocytes and thrombocytes. An example of processed blood product is an enriched blood product comprising enriched erythrocytes, enriched thrombocytes or enriched erythrocytes and thrombocytes. For instance, an erythrocyte sample obtained following centrifugation of whole blood is an enriched erythrocyte blood product, a buffy coat obtained following centrifugation of whole blood is an enriched thrombocyte blood product, whereas removal of the blood plasma following centrifugation of whole blood leaves a combined enriched erythrocyte and thrombocyte blood product.

Another aspect of the invention relates to PEG-phospholipid molecules for use in selective masking of surface antigens of an organ transplant comprising erythrocytes, thrombocytes and/or endothelial cells from a donor to inhibit antibody binding to the surface antigens and/or antibody-mediated rejection (AMR) in connection with transplantation of the organ transplant into an uncrossmatched or incompatible recipient.

A related aspect of the invention defines use of PEG-phospholipid molecules for the manufacture of a medicament for inhibiting antibody binding to the surface antigens and/or antibody-mediated rejection (AMR) in connection with transplantation of an organ transplant comprising erythrocytes, thrombocytes and/or endothelial cells having surface antigens selectively masked by the PEG-phospholipid molecules into an uncrossmatched or incompatible recipient.

The selective masking of the surface antigens of erythrocytes, thrombocytes and/or endothelial cells by the PEG-phospholipid molecules of the embodiments thereby inhibit agglutination and AMR by inhibiting antibody binding to the surface antigens in connection with transplantation of the organ transplant into an uncrossmatched or incompatible recipient. Hence, this aspect of the invention relates to PEG-phospholipid molecules for use in selective masking of surface antigens of an organ transplant comprising erythrocytes, thrombocytes and/or endothelial cells from a donor to inhibit agglutination and/or antibody-mediated rejection (AMR) in connection with transplantation of the cell or organ transplant into an uncrossmatched or incompatible recipient.

Antibody-mediated rejection (AMR), also known as B-cell-mediated or humoral rejection, is a significant complication after organ transplantation, such as kidney transplantation. Although fewer than 10% of kidney transplant patients experience AMR, as many as 30% of these patients experience graft loss as a consequence. AMR is mediated by antibodies against an allograft, especially anti-HLA antibodies and A/B blood type antibodies, and results in histologic changes in allograft vasculature that differ from cellular rejection (T-cell-mediated rejection). Hence, AMR is a separate disease process as compared to cellular rejection. AMR is initially characterized by microvascular inflammation, endothelial injury, and serological evidence of donor-specific antibodies (DSA). The AMR symptoms may progress into transplant glomerulopathy, a form of advanced glomerular injury and remodeling.

The standard of care for AMR includes plasmapheresis and intravenous immunoglobulin that remove and neutralize antibodies, respectively. Agents targeting B cells (rituximab and alemtuzumab), plasma cells (bortezomib), and the complement system (eculizumab) have also been used to treat AMR in kidney transplant recipients.

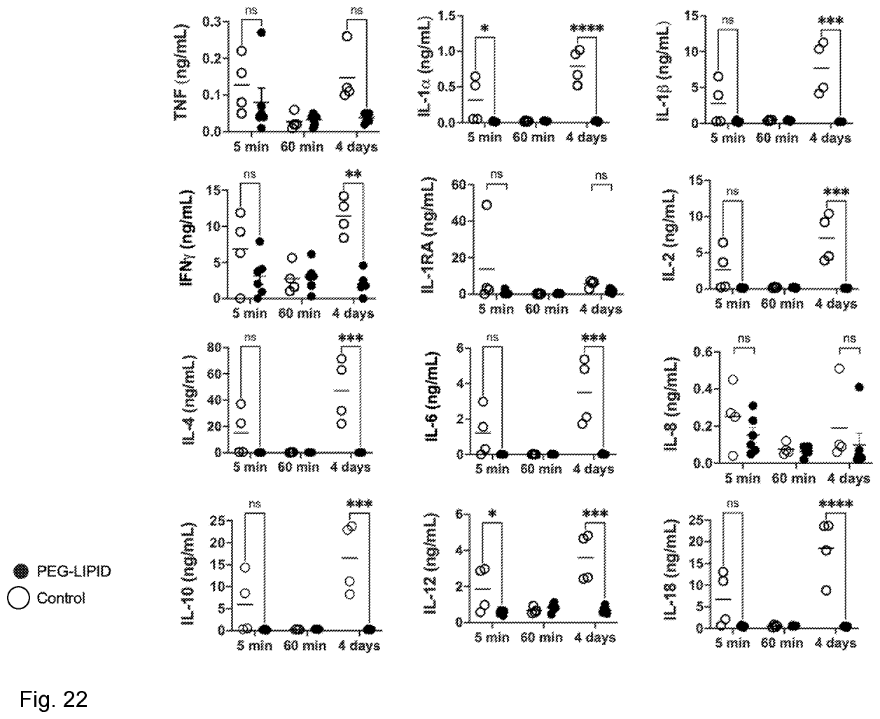

AMR is thereby a rejection process different from the initial cellular rejection driven by the innate immune system and that takes place immediately following transplantation of the organ graft. Hence, AMR follows the initial cellular rejection process. The PEG-phospholipids of the present invention has a half-life in pigs of about 14 h, i.e., well before the AMR process is initiated. However, as shown in the experimental section, the PEG-phospholipid molecules of the present invention were able to suppress cytokines even at 4 days following transplantation. For instance, as shown in FIG. 22, at 60 min following transplantation there is not any significant difference between untreated control kidneys and PEG-phospholipid treated kidneys for several cytokines including IL-1α, IL-1β, IFNγ, IL-2, IL-4, IL-6, IL-01, IL-12 and IL-18. However, at 4 days following kidney transplantation, the recipients receiving the PEG-phospholipid treated kidneys had significantly lower levels of these cytokines as compared to the recipients receiving the untreated control kidneys. This was highly surprising since at 4 days following transplantation the PEG-phospholipid molecules, having a half-life of merely 14 hours, are gone from the treated kidneys and still their effects remain.

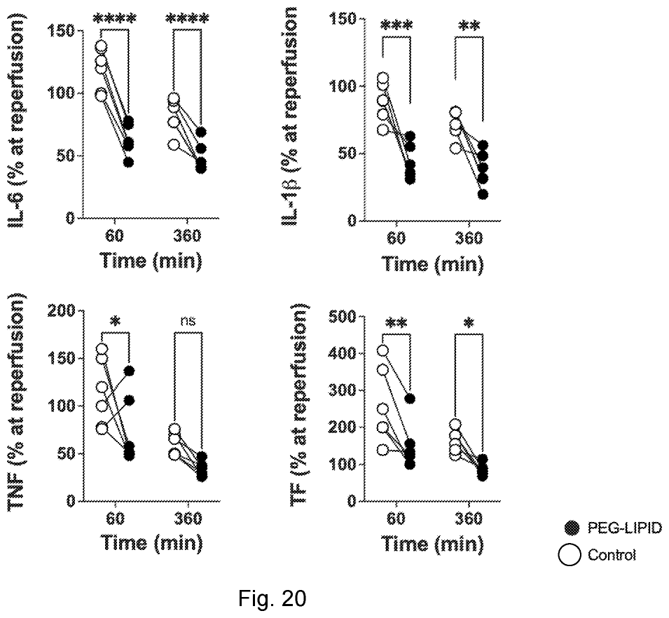

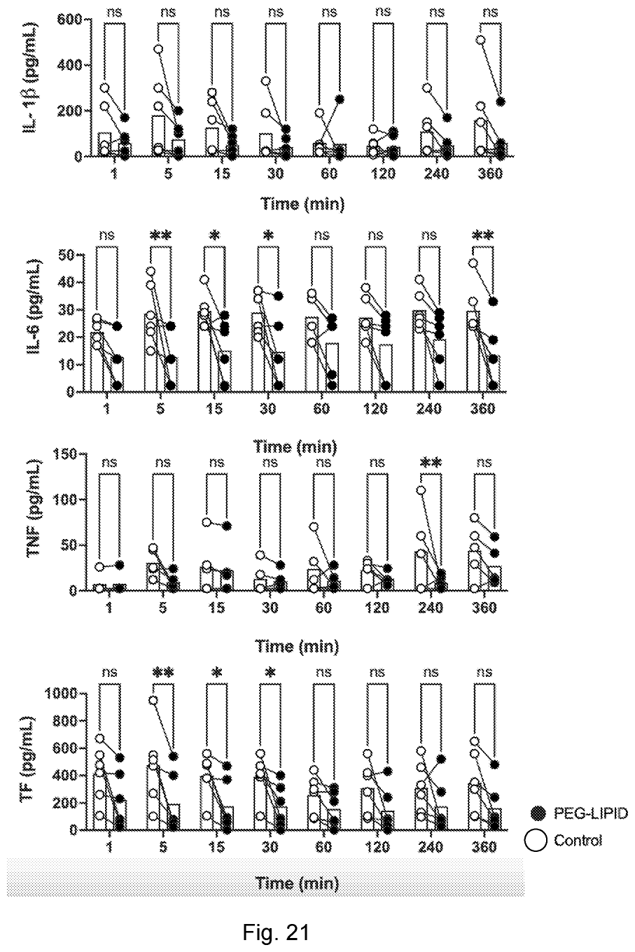

These long-term effects seen by treating organ transplants with the PEG-phospholipid mean that the PEG-phospholipids protect against the adaptive immune system and AMR. The consistent pronounced inhibition of IL-6 supports this notion, since IL-6/IL-6R signaling inhibition is considered a novel therapeutic option for the prevention and treatment of allograft injury and AMR. Evidence from clinical trials supports the use of IL-6 blockade for desensitization and treatment of AMR in kidney transplantation (Curr Transplant Reports 8:1-14 (2021), J Am Soc Nephrol 20:1032-1040 (2009), Transplantation 101:32-44 (2017)). As is shown in FIG. 22, and also in FIGS. 20-21, the PEG-phospholipid molecules led to a significant inhibition of IL-6, which is important in targeting and treating AMR.

Uncrossmatched recipient as used herein refers to a recipient, for whom no cross-matching has been performed between the donor of the blood product or organ transplant and the recipient. Incompatible recipient as user herein refers to a recipient that is incompatible with at least one surface antigen on the erythrocytes and/or thrombocytes in the blood product, i.e., a recipient having antibodies against at least one surface antigen on the erythrocytes and/or thrombocytes, or at least one surface antigen on the erythrocytes, thrombocytes and/or endothelial cells in the organ transplant.

The present invention is not limited to allogeneic transplantation of organ transplants, i.e., organ allotransplants. In clear contrast, the present invention also provides advantages in connection with xenogeneic transplantation of organ transplants, i.e., organ xenotransplants. For instance, organ transplants from a donor of one mammalian species, typically non-human mammalian species, could be treated with PEG-phospholipid molecules of the invention to selective mask surface antigens of the organ transplant prior to transplantation into a recipient of another mammalian species, typically a human.

In an embodiment, the recipient is an uncrossmatched recipient. In another embodiment, the recipient is an incompatible recipient.

An organ transplant as used herein includes a complete organ or tissue or a portion of an organ that is to be transplanted into a recipient body. Illustrative, but non-limiting, examples of organ transplants include kidney, liver, pancreas, heart, lung, uterus, urinary bladder, thymus and intestine, including portions thereof.

Agglutination as used herein refers to the clumping of cells that occurs if an antigen is mixed with its corresponding antibody. For instance, when an uncrossmatched or incompatible recipient is given blood transfusion, antibodies in the recipient may react with surface antigens on erythrocytes and/or thrombocytes and as a result erythrocytes and/or thrombocytes clump up and stick together causing them to agglutinate.

Selective masking of surface antigens on erythrocytes by PEG-phospholipid molecules of the embodiments includes selective masking of blood group antigens. Such blood group antigens are preferably selected from the group consisting of A antigens and B antigens. For instance, treating a blood product comprising erythrocytes from a donor having blood group A to selectively mask the A antigens on the erythrocytes would result in a blood product that could be given to not only a type A recipient but also to type B, type AB or type 0 recipients with no or at least significantly reduced risk of having any initial agglutination reaction in connection with blood transfusion. Hence, in an embodiment, the surface antigens are blood group antigens. In a particular embodiment, the surface antigens are selected from the group consisting of A antigen and B antigen.

In another embodiment, the surface antigens are Rhesus factors. In a particular embodiment the surface antigens are selected from the group consisting of RhD, RhC, Rhc, RhE and Rhe.

Hence, in an embodiment, the PEG-phospholipid molecules selectively mask a blood group antigen, such as A antigen and/or B antigen. In another embodiment, the PEG-phospholipid molecules selectively mask a Rhesus facture, such as RhD, RhC, Rhc, RhE and/or Rhe. In a further embodiment, the PEG-phospholipid molecules selectively mask a blood group antigen, such as A antigen and/or B antigen, and selectively mask a Rhesus facture, such as RhD, RhC, Rhc, RhE and/or Rhe.

In a preferred embodiment, the PEG-phospholipid molecules of the embodiments selectively mask any blood group antigens and any Rhesus factors present on the cell surface of erythrocytes in the blood product.

Correspondingly, endothelial cells of an organ transplant comprise surface antigens in the form of blood group antigens. Accordingly, the PEG-phospholipid molecules of the embodiments can selectively mask any blood group antigens present on the cell surface of endothelial cells in an organ transplant. Furthermore, endothelial cells comprise surface antigens in the form of human leukocyte antigens (HLAs). In an embodiment, the PEG-phospholipid molecules of the embodiments can therefore selectively mask any HLA present on the cell surface of endothelial cells in an organ transplant. In a particular embodiment, the HLAs are selected from the group consisting of HLA A, HLA B and HLA C, which are the major antigens of major histocompatibility complex (MHC) class I. This MHC class I also comprise minor HLA antigens in the form of HLA E, HLA F and HLA G. The MHC class I proteins form a functional receptor on most nucleated cells of the body, whereas MHC class II proteins, such as HLA-DP, HLA-DG, HLA-DR, HLA-DM and HLA-DO, only occur on antigen-presenting cells, B cells and T cells.

Furthermore, PEG-phospholipid molecules of the embodiments can selectively mask CL-11. In transplantation, CL-11 expression in the organ graft, such as kidney graft, recognizes ischemic cells and contributed to complement activation via the lectin (LP) pathway. Also other recognition molecules of the LP, such as MBL, have been associated with complement activation in connection with organ transplantation. Hence, selectively masking such cell membrane proteins on the endothelial with a PEG-phospholipid coating shielding off the endothelial membranes of organ transplants will reduce complement activation in connection with organ transplantation.

Thrombocytes comprise surface antigens referred to as human platelet antigens (HPAs). In an embodiment, the surface antigens are HPAs. In a particular embodiment, the surface antigens are selected from the group consisting of HPA-1, HPA-2, HPA-3, HPA-4, HPA-5, HPA6, HPA-9 and HPA-15. In another particular embodiment, the surface antigens are selected from the group consisting of HPA-1, HPA-2, HPA-3, HPA-4, HPA-5, HPA6 and HPA-15. In a further particular embodiment, the surface antigens are HPA-1.

Treating a blood product comprising both erythrocytes and thrombocytes or a cell or organ transplant comprising thrombocytes in addition to erythrocytes and/or endothelial cells result in a selective masking of both blood group antigens, Resus factors and HPAs by the PEG-phospholipid molecules.

In an embodiment, the PEG-phospholipid molecules have an average extracellular length, when anchored in a cell membrane, selected within an interval of from 4 nm up to 8 nm. In a particular embodiment, the PEG-phospholipid molecules have an average extracellular length, when anchored in a cell membrane, selected within an interval of from 5 nm up to 7 nm, and preferably about 6 nm.

Average extracellular length as used herein indicates that individual PEG-phospholipid molecules may have a length extending from the cell surface when anchored in the cell membrane that is longer or shorter than the average extracellular length. However, the average extracellular length is the average or mean of the individual lengths of the PEG-phospholipid molecules.

The above-described average extracellular length is sufficient long to mask blood group antigens, Rhesus factors, HLAs and HPAs but not sufficiently long to effectively mask extracellular or transmembrane molecules and proteins in the cell membrane having an extracellular domain or portion that is significantly longer or larger than this average extracellular length of the PEG-phospholipid molecules.

In an embodiment, the PEG-phospholipid molecules have a formula (I):

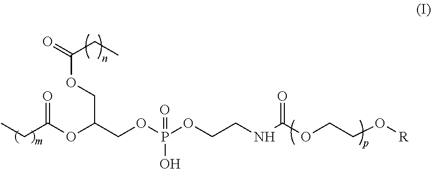

In an embodiment, n, m are integers independently selected within the range of from 10 up to 16. The parameters n, m are preferably independently 10, 12, 14 or 16, and more preferably n=m=14.

In an embodiment, p is selected so that the PEG chain or domain has an average molecular weight selected within the range of from 1 000 Da up to 40 000 Da. The parameter p is preferably selected so that the PEG chain has and average molecular weight from 3 000 up to 10 000 Da and more preferably about 5 000 Da, such as from 4 500 Da up to 5 500 Da, from 4 600 Da up to 5 400 Da, from 4 700 Da up to 5 300 Da, from 4 800 Da up to 5 200 Da or from 4 900 Da up to 5 100 Da.

Average molecular weight as defined herein indicates that individual PEG-phospholipid molecules may have a molecular weight different from this average molecular weight but that the average molecular weight represents the mean molecular weight of the PEG-phospholipid molecules. This further implies that there will be a natural distribution of molecular weights around this average molecular weight for a PEG-phospholipid sample.

The end group R is, in an embodiment, selected from the group consisting of H, methyl (CH₃), and C1-C4 alkyl amine. In an embodiment, the C1-C4 alkyl amine is n-propylamine. In a preferred embodiment, the end group R is methyl.

In an embodiment, the PEG-phospholipid molecule is N-(methylpolyoxyethylene oxycarbonyl)-1,2-dipalmitoyl-sn-glycero-3-phosphoethanolamine.

In another embodiment, the end group R could comprise a maleimide group, a biotin group, a streptavidin group, an avidin group or a functionalized molecule. In the case of a functionalized molecule this is preferably selected from the group consisting of a complement inhibitor, a coagulation inhibitor, a platelet inhibitor, a molecule capable of binding a complement inhibitor, a molecule capable of binding a coagulation inhibitor, a molecule capable of binding a platelet inhibitor, and a mixture thereof.

In such a case, the maleimide group, the biotin group, the streptavidin group, the avidin group or the functionalized molecule is preferably attached to the PEG-phospholipid molecule of formula (I) by a linker. An illustrative, but non-limiting, example of such a linker is —CH₂CH₂CH₂NHC(O)CH₂CH₂—. For instance, an end group R comprising such a linker and a maleimide groups is presented in formula (II):

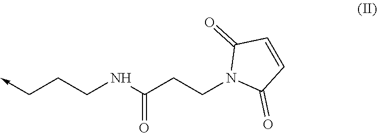

The functionalized molecule could be attached to the PEG-phospholipid molecule of formula (I) via a linker. Hence, in this embodiment, the end group R has the general formulas of L-(FM), wherein L represents the linker and (FM) represents the functionalized molecule.

Illustrative, but non-limiting, examples of complement inhibitors include Factor H; C4b-binding protein (C4BP); N-terminal 4 to 6 short consensus repeats (SCRs) of complement receptor 1 (CR1), also known as C3b/C4b receptor or cluster of differentiation 35 (CD35); CD46 complement regulatory protein (CD46), also known as membrane co-factor protein (MCP); and complement decay-accelerating factor (DAF), also known as CD55.

An illustrative, but non-limiting, example of coagulation inhibitors includes heparin.

An illustrative, but non-limiting, example of a platelet inhibitor is an adenosine diphosphate (ADP) degrading enzyme, such as an apyrase and ectonucleoside triphosphate diphosphohydrolase-1 (NTPDase1), also known as CD39.

Illustrative, but non-limiting examples of a molecule capable of binding a complement inhibitor is a Factor H-binding peptide, such as 5C6 (Nilsson et al., Autoregulation of thromboinflammation on biomaterial surfaces by a multicomponent therapeutic coating. Biomaterials. 2013, 34 (4): 985-994), and a C4BP binding peptide, such as Streptococcus M protein-derived peptide M2-N, M4-N or M22-N (Engberg et al., Inhibition of complement activation on a model biomaterial surface by streptococcal M protein-derived peptides. Biomaterials. 2009, 30 (13): 2653-2659).

An illustrative, but non-limiting example of a molecule capable of binding a coagulation inhibitor is a heparin-binding peptide (Asif et al., Heparinization of cell surfaces with short peptide-conjugated PEG-phospholipid regulates thromboinflammation in transplantation of human MSCs and hepatocytes. Acta Biomateriala. 2016, 35:194-205).

Hence, in an embodiment, the functionalized molecule is selected from the group consisting of a heparin-binding peptide, N-terminal 4 to 6 SCRs of CR1, CD46, DAF, a Factor FI binding molecule, an ADP degrading enzyme, and a mixture thereof.

The fatty acid chains of the PEG-phospholipid molecule may be saturated. At least one or both fatty acid chains may alternatively be unsaturated, i.e., comprise at least one —CH═CH— group, at least one —C≡C— group or a combination thereof. Each fatty acid chain could be straight or branched.

In an embodiment, the PEG-phospholipid molecules have an average molecular weight as determined by gel permeation chromatography selected within an interval of from 5 to 7 kDa. In a particular embodiment, the PEG-phospholipid molecules have an average molecular weight as determined by gel permeation chromatography selected within an interval of from 5.5 to 6.6 kDa. In a preferred embodiment, the PEG-phospholipid molecules have an average molecular weight as determined by gel permeation chromatography selected within an interval of from 5.8 to 6.0 kDa, such as about 5.9 kDa.

In an embodiment, the lipid part or domain of the PEG-phospholipid molecules is a phospholipid part or domain.

In an embodiment, the lipid part or domain of the PEG-phospholipid molecules is 1,2-dipalmitoyl-sn-glycerol-3-phosphatidylethanolamine (DPPE). Other, non-limiting, examples of the lipid part or domain of the PEG-phospholipid molecules include 1,2-dimyristoyl-sn-glycero-3-phosphorylethanolamine (DMPE), also referred to as 1,2-ditetradecanoyl-sn-glycero-3-phosphoethanolamine and [(2R)-3-[2-aminoethoxy(hydroxy)phosphoryl]oxy-2-tetradecanoyloxypropyl]tetradecanoate, and 1,2-distearoyl-sn-glycero-3-phosphorylethanolamine (DSPE).

In an embodiment, the PEG part or domain of the PEG-phospholipid molecules has an average degree of polymerization (DP) selected within an interval of from 105 to 125. In a particular embodiment, the PEG part or domain of the PEG-phospholipid molecules has an average DP selected within an interval of within an interval of from 110 to 120. In a preferred embodiment, the PEG part or domain of the PEG-phospholipid molecules has an average DP selected within an interval of from 114 to 116, such as about 115.

In an embodiment, the PEG-phospholipid molecules comprise at least one sulfated glycosaminoglycan. In a particular embodiment, the PEG-phospholipid molecule comprises a KₙC and/or CKₙ link interconnecting the at least one sulfated glycosaminoglycan and the PEG-phospholipid. In this particular embodiment, C is cysteine, K is lysine and n is zero or a positive integer equal to or smaller than 20, preferably n is selected within the interval of from 0 to 15, and more preferably n is selected within the interval of from 0 to 10.

In an embodiment, the sulfated glycosaminoglycan is fragmented heparin. In a particular embodiment, the fragmented heparin has an average molecular weight selected within the interval of from 2.5 kDa to 15 kDa, preferably within the interval of from 5 kDa to 10 kDa, and more preferably within the interval of from 7 kDa to 9 kDa.

In an embodiment, any free amino groups in the sulfated glycosaminoglycan-PEG-phospholipid are converted into carboxylic groups.

Such PEG-phospholipid molecules can be produced by mixing a cation-PEG-phospholipid comprising at least one amino group with a sulfated glycosaminoglycan comprising at least one carbonyl group, preferably at least one aldehyde group, to form a Schiff base intermediate. A reducing agent is added to the Schiff base intermediate to form a sulfated glycosaminoglycan-PEG-phospholipid.

For instance, α-N-hydroxysuccinimidyl-ω-maleimidyl PEG (NHS-PEG-Mal), triethylamine and 1,2-dipalmitoyl-sn-glycerol-3-phosphatidylethanolamine (DPPE) are mixed in dicholoromethane. A next step comprises precipitating the resulting maleimide-conjugated PEG-phospholipid by adding diethyl ether to the mixture of NHS-PEG-Mal and DPPE in dichloromethane.

The preferred sulfated glycosaminoglycan, i.e., fragmented heparin, preferably comprises at least one carbonyl group, more preferably at least one aldehyde group. Such a fragmented heparin can be obtained by mixing an acidic solution and a sodium nitrite (NaNO₂) aqueous solution to form a mixed solution. The pH of the mixed solution is adjusted within an interval of from 2 up to 6, preferably from 3 up to 5, and more preferably 4. Heparin, preferably heparin sodium, is added to the mixed solution to form a heparin solution and the pH of the heparin solution is adjusted within an interval of from 6 to 8, preferably from 6.5 to 7.5 and more preferably to 7 to form the fragmented heparin comprising at least one carbonyl group. The fragmented heparin comprising at least one carbonyl group is optionally dialyzed against water and lyophilizing the fragmented heparin comprising at least one carbonyl group.

An illustrative example of a reducing agent that is added to Schiff base is sodium cyanoboronhydride.

In an embodiment, the PEG-phospholipid molecules are separate or individual PEG-phospholipid molecules when anchored in the cell membrane of erythrocytes and/or thrombocytes, or of an organ transplant. This means that the PEG-phospholipid molecules are not interconnected or linked to each other with any interconnecting molecules or linker, such as poly(vinyl alcohol) (PVA) or multiple-arm-PEG-SH, with the purpose of interconnecting individual PEG-phospholipid molecules and forming a layered surface masking or camouflage on the cell surface.

A further aspect of the invention relates to a blood product comprising erythrocytes and/or thrombocytes and PEG-phospholipid molecules anchored in the cell membrane of the erythrocytes and/or thrombocytes and masking surface antigens of the erythrocytes and/or thrombocytes. A PEG-chain of the PEG-phospholipid molecules has an average molecular weight selected within the range of from 3 000 up to 10 000 Da.

Yet another aspect of the invention relates to an in vitro method of treating erythrocytes and/or thrombocytes. The method comprises selective masking surface antigens of erythrocytes and/or thrombocytes by adding in vitro PEG-phospholipid molecules to the erythrocytes and/or thrombocytes. A PEG-chain of the PEG-phospholipid molecules has an average molecular weight selected within the range of from 3 000 up to 10 000 Da.

In blood transfusion services, recipients are traditionally screened for irregular antibodies in order to be able to select suitable compatible blood. These irregular antibodies are pre-existing antibodies that a recipient may have already developed following previously blood transfusions and/or following a previous childbirth. Generally, irregular antibodies are all non-ABO antibodies, although the main use of the term is for non-ABO isoantibodies that may cause incompatibility in blood transfusions. Irregular antibodies are most commonly of the IgG type, and they appear first after exposure to foreign antigens. Illustrative, but non-limiting, examples of such irregular antibodies include anti-c, anti-Cw, anti-D, anti-E, anti-Fya, anti-Jka, anti-Kell, anti-Kpb, anti-Lea, anti-Leb (Lewis), anti-Lua, anti-Lub, anti-M, anti-N, anti-P1, anti-Public, anti-S, anti-s and auto-Pap antibodies. Since the patient can have more than one of these irregular antibodies, the identification of antibodies can be difficult. In order to simplify the identification, selective masking of surface antigens would make the identification easier since at least a portion of the surface antigens present on the surface of erythrocytes could be masked and hidden by the PEG-phospholipid molecules of the embodiments.

Hence, an aspect of the invention relates to an irregular antibody screening method. The method comprises adding PEG-phospholipid molecules to a blood sample from a subject to selectively mask surface antigens on erythrocytes present in the blood sample. The method also comprises screening for irregular antibodies bound to erythrocytes present in or obtained from the blood sample. A PEG-chain of the PEG-phospholipid molecules has an average molecular weight selected within the range of from 3 000 up to 10 000 Da.

The initial step in the method selectively masks A and/or B antigens present on the surface on the erythrocytes but do not selectively mask all surface antigens, against which irregular antibodies can be raised and bond to. The treated blood sample could then be subject to an irregular antibody screening test. Screening of antibodies is often through performing a Coombs test, which is also known as an antiglobulin test or red blood cell antibody screening. There are two types of Coombs test: direct and indirect. The direct Coombs test involves collecting erythrocytes (red blood cells) from a blood sample taken from a subject and preferably washing the erythrocytes. The collected and optionally washed erythrocytes are then incubated with anti-human antibodies (Coombs reagent), which bind to the Fc region of any irregular antibodies present in the blood sample and bound to surface antigens on the erythrocytes. If there are any irregular antibodies present in the blood sample, the anti-human antibodies form links between erythrocytes by binding to the human irregular antibodies on the erythrocytes causing an erythrocyte agglutinate. In the direct Coombs test, PEG-phospholipid molecules of the embodiments are added to the blood sample prior to collecting erythrocytes and/or, preferably, added to the erythrocytes collected from the blood sample.

The indirect Coombs test involves mixing a blood sample from a donor with a serum sample from a recipient. Any irregular antibodies present in the serum sample from the recipient may bind to surface agents present on the erythrocytes in the blood sample from the donor thereby forming antibody-antigen complexes. Anti-human antibodies (Coombs reagent) are added to the mixture causing erythrocyte agglutinate as in the direct Coombs test if the serum sample from the recipient contained any irregular antibodies capable of binding to surface antigens on erythrocytes in the blood sample from the donor. In the direct Coombs test, PEG-phospholipid molecules of the embodiments may be added to the blood sample of the donor, preferably prior to mixing with the serum sample from the recipient.

The irregular antibody screening method is an in vitro screening method.

Another aspect of the invention relates to a blood transfusion method. The method comprises adding PEG-phospholipid molecules to a blood product from a donor to selectively mask surface antigens on erythrocytes and/or thrombocytes present in the blood product. The method also comprises transfusing the blood product into an uncrossmatched or incompatible recipient. A PEG-chain of the PEG-phospholipid molecules has an average molecular weight selected within the range of from 3 000 up to 10 000 Da.

The PEG-phospholipid molecules of the embodiments can be added to the blood product or to the cells in vitro in a concentration selected to efficiently selective mask surface antigens on the erythrocytes and/or thrombocytes, such as in the blood product. Hence, in an embodiment, the final concentration of PEG-phospholipid molecules in the blood product may be dependent on the amount of on the erythrocytes and/or thrombocytes in the blood product.

In a typical embodiment, the PEG-phospholipid molecules could be added to the cells, blood product or cell transplant to achieve a final concentration of PEG-phospholipid molecules in the cells, blood product or cell transplant selected within an interval of from 0.25 mg/ml up to 25 mg/ml, preferably from 0.25 mg/ml up to 10 mg/ml. In a particular embodiment, the final concentration of PEG-phospholipid molecules in the cells, blood product or cell transplant is from 0.25 mg/ml up to 5 mg/ml, preferably from 0.5 mg/ml up to 4 mg/ml or from 1 mg/ml up to 3 mg/ml. In a preferred embodiment, the final concentration of PEG-phospholipid molecules in the cells, blood product or cell transplant is from 1.5 mg/ml up to 2.5 mg/ml, such as about 2 mg/ml.

The PEG-phospholipid molecules can be added in the form of a solution of PEG-phospholipid molecules in a solvent, preferably an aqueous solvent. Non-limiting, but illustrative, examples of such aqueous solvents include saline, buffer solutions and organ preservation solutions.

The above-described embodiments of surface antigens and PEG-phospholipids also apply to these aspects of the embodiments.

In transplantation, in particular in living donor kidney transplantation, ABO incompatibility is a hinder for effective transplantation. In order to perform the transplantation under these conditions, the anti-ABO antibodies are traditionally removed during several weeks using plasmaphereses and anti-CD20 antibodies. This pre-treatment over several weeks is very time consuming and cumbersome. There is therefore a need for treatment of transplants and grafts so that, for instance, ABO antigens are masked, thereby facilitating transplantation.

An organ transplant can be treated with PEG-phospholipid molecules of the embodiments by administering the PEG-phospholipid molecules into the vascular system of the organ transplant and/or by submerging the organ transplant into a solution, preferably an organ preservation solution, comprising PEG-phospholipid molecules.

For instance, a solution comprising PEG-phospholipid molecules is ex vivo infused into a vascular system and, optionally into a parenchyma, of the organ transplant. The solution comprising PEG-phospholipid molecules is preferably ex vivo incubated in the vascular system, and optionally the parenchyma, to enable coating at least a portion of the endothelial lining of the vascular system, and preferably of the parenchyma, with the PEG-phospholipid molecules.

In an embodiment, the ex vivo incubating step comprises ex vivo incubating the solution comprising PEG-phospholipid molecules in the vascular system, and optionally the parenchyma, to enable coating at least a portion of the endothelial lining of the vascular system, and preferably of the parenchyma, with the PEG-phospholipid molecules while keeping the organ transplant submerged in an organ preservation solution, preferably an organ preservation solution comprising PEG-phospholipid molecules.

Thus, the ex vivo treatment of the organ transplant comprises introducing PEG-phospholipid molecules into the vascular system of the organ transplant and therein allow the PEG-phospholipid molecules to interact with and bind to the cell membranes of the endothelium and the parenchyma.

The interaction between the PEG-phospholipid molecules with the lipid bilayer membrane of the endothelium and optionally of the parenchyma, such as renal parenchyma in the case of a kidney, is preferably taking place ex vivo while the organ transplant is submersed or submerged in an organ preservation solution, preferably an organ preservation solution comprising PEG-phospholipid molecules.

In a particular embodiment, the organ transplant is first ex vivo infused with the solution comprising PEG-phospholipid molecules into the vascular system and, optionally into the parenchyma, of the organ transplant. This ex vivo infusion is advantageously taking place as early as possible following explanting and removing the organ from the donor body. The perfused organ is then submerged in the organ preservation solution, preferably comprising PEG-phospholipid molecules, and kept therein, preferably at reduced temperature such as about 4° C.

In another particular embodiment, the organ is first submerged into the organ preservation solution, preferably comprising PEG-phospholipid molecules, and then the solution comprising PEG-phospholipid molecules is ex vivo infused into the vascular system, and optionally into the parenchyma, of the organ. This ex vivo infusion can be performed while keeping the organ submerged in the organ preservation solution, preferably comprising PEG-phospholipid molecules. Alternatively, the organ is temporarily removed from the organ preservation solution to perform the ex vivo infusion and is then put back into the organ preservation solution, preferably comprising PEG-phospholipid molecules.

In an embodiment, the method also comprises ex vivo infusing an organ preservation solution into the vascular system to flush away non-bound PEG-phospholipid molecules from the vascular system. Hence, non-bound PEG-phospholipid molecules are preferably washed away in one or multiple, i.e., at least two, wash steps using an organ preservation solution.

In an embodiment, ex vivo infusing the solution comprising PEG-phospholipid molecules comprises ex vivo clamping one of an artery and a vein of the vascular system. This embodiment also comprises ex vivo infusing the solution comprising PEG-phospholipid molecules into the other of the artery and the vein and ex vivo clamping the other of the artery and the vein.

In another embodiment, the solution with PEG-phospholipid molecules is infused into an artery (or vein) of the vascular system of the organ transplant until the solution appears at a vein (or artery) of the organ transplant. This confirms that the solution with PEG-phospholipid molecules has filled the vascular system. At that point, the artery and vein are clamped.

The solution comprising PEG-phospholipid molecules can be added either through a vein or through an artery. In a particular embodiment, the solution is infused into an artery. In such a particular embodiment, the optional, initial clamping is then preferably done of a vein of the vascular system.

The solution comprising PEG-phospholipid molecules is preferably ex vivo incubated in the vascular system for a period of time from 10 minutes up to 48 hours to enable the PEG-phospholipid molecules to hydrophobically interact with the cell membranes of the endothelium and thereby coat at least a portion of the vascular system of the organ transplant. The ex vivo incubation is preferably performed from 20 minutes up to 36 hours and more preferably from 30 minutes up to 24 hours, such as from 30 minutes up to 12 hours, up to 8 hours, up to 4 hours or up to 1 hour.

The amount of solution comprising PEG-phospholipid molecules infused into the vascular system depends on the type of the organ and the size of the organ (adult vs. child). Generally, the volume of the solution should be sufficient to fill the vascular system of the organ. In most practical applications, from 5 ml up to 500 ml of the solution comprising PEG-phospholipid molecules is ex vivo infused into the vascular system. In a preferred embodiment, from 5 ml up to 300 mL and preferably from 10 mL up to 250 ml solution comprising PEG-phospholipid molecules is ex vivo infused into the vascular system.

In an embodiment, the solution comprises from 0.25 mg/ml up to 25 mg/ml, preferably from 0.25 mg/ml up to 10 mg/ml PEG-phospholipid molecules. In a particular embodiment, the solution comprises from 0.25 mg/ml up to 5 mg/ml, preferably from 0.5 mg/ml up to 4 mg/ml or from 1 mg/ml up to 3 mg/ml PEG-phospholipid molecules. In a preferred embodiment, the solution comprises from 1.5 mg/ml up to 2.5 mg/ml, such as about 2 mg/ml, PEG-phospholipid molecules.

The above-described concentrations of PEG-phospholipid molecules can also be used for the organ preservation solution comprising PEG-phospholipid molecules.

The PEG-phospholipid molecules are preferably administered in the form of a PEG-phospholipid solution. The solution comprising the PEG-phospholipid molecules could, for instance, be saline, an aqueous buffer solution or an organ preservation solution. Illustrative, but non-limiting, examples of aqueous buffer solutions that could be used include phosphate-buffered saline (PBS) and a citrate solution.

Illustrative, but non-limiting, examples of organ preservation solutions that can be used according to the embodiments include a histidine-tryptophan-ketoglutarate (HTK) solution, a citrate solution, a University of Wisconsin (UW) solution, a Collins solution, a Celsior solution, a Kyoto University solution and an Institut Georges Lopez-1 (IGL-1) solution. In a particular embodiment, the organ preservation solution is a HTK solution.

The above-described embodiments of surface antigens and PEG-phospholipids also apply to this aspect of the embodiments.

### EXAMPLES

### Example 1

This Example investigated the protective effect of a particular PEG-phospholipid by shielding different cell types with PEG-phospholipid coating and assessing the blocking effect it has on the binding of antibodies to surface antigens.

### Materials and Methods

### Production Procedure

Polyethylene glycol-phospholipid with PEG of 5 kDa (PEG-phospholipid) or free PEG of 5 kDa was used in this Example (NOF Corp., Japan). Non-conjugated PEG-phospholipid (SUNBRIGHT® PP-050CN, N-(methylpolyoxyethylene oxycarbonyl)-1,2-dipalmitoyl-sn-glycero-3-phosphoethanolamine, sodium salt, NOF Corp., Japan) was used as it is. Free PEG (5 kDa) was prepared from α-N-hydroxysuccinimidyl-ω-maleimidyl poly(ethylene glycol) (NHS-PEG-Mal, Mw: 5,000, NOF), which was reacted with glycine and cysteine to deactivate NHS and the maleimide group, followed by purification on a spin column.

### Cell Type

Human erythrocytes were isolated from healthy human donors. They were pre-selected for specific blood types and only individuals exposing blood type B antigens were used. Human blood was drawn from the donors using a vacuum blood collection tube (5 mL, EDTA-2Na treated, TERUMO Co., Tokyo, Japan).

After plasma and buffy coat were removed by centrifugation (15 min, 2500×g, 3 times), the erythrocytes were re-suspended in cold PBS.

Caucasian acute lymphoblastic leukemia cell line (CCRF-CEM) was purchased from the Health Science Research Resources Bank (Tokyo, Japan). CCRF-CEM cells were cultured (at 37° C., 5% CO₂) in RPMI-1640 medium (containing 10% heat inactivated fetal bovine serum, Penicillin 50 IU/mL, and Streptomycin 50 μg/mL).

Coating Effect of PEG-Phospholipid from Antibody Reaction Using Human Erythrocytes

This Example was approved by ethical committee of the University of Tokyo (KE17-13). Human red blood cells (RBC) were isolated from whole blood of a type-B donor and suspended in phosphate-buffered saline (PBS). A cell pellet of RBCs (5×10⁷ cells) was incubated with PEG-phospholipid (20 μl, 2 mg/ml in PBS) or PBS (50 μl, as control) for 30 min at room temperature (RT, 20-25° C.) with gentle mixing. After wash with PBS by centrifugation (120×g, 4° C., 1 min) twice, Alexa488-labeled anti-blood group B antigen antibody (10 μl, used without dilution, Blood Group B antigen antibody [HEB-29], GeneTex) or PBS (10 μl) was mixed with the treated RBCs for 10 min at RT. After washing with PBS by centrifugation (120×g, 4° C., 1 min), RBC were re-suspended with PBS (1 ml) for flow cytometry measurements. All samples were analyzed on an Accuri C6 flow cytometer (BD), additional controls in the form of isotype control and unstained control were ran in parallel. Post analysis processing was carried out in FlowJo (BD).

Coating Effect of PEG-Phospholipid from Antibody Reaction Using CCRF-CEM

CCRF-CEM were collected from cell culture medium (1×10⁶ cells) and washed with PBS by centrifugation (250×g, 3 min, RT). A cell pellet of CCRF-CEM (1×10⁶ cells) was incubated with PEG-phospholipid (50 μl, 2 mg/ml in PBS), 5 kDa unconjugated PEG (50 μl, 2 mg/ml in PBS) or PBS (50 μl, as control) for 30 min at RT with gentle mixing. After washing with PBS by centrifugation (200×g, 4° C., 3 min) twice, FITC-labeled three antibodies, FITC-anti-human CD52 from BioLegend (400 μg/ml, 40 μl), FITC-anti-human CD4 from BioLegend (200 μg/ml, 40 μl), or Alexa488-labelled anti-human CD8 from GeneTex (200 μg/ml, 40 μl, Alexa488 was labelled by the labelling kit) or PBS (40 μl) was mixed with the treated CCRF-CEM for 15 min at RT. As a control, CCRF-CEM was treated with 2 mg/ml PEG (5 kDa, in PBS) solution and additionally incubated with FITC- or Alexa488-labeled antibodies. After washing with PBS by centrifugation (200×g, 4° C., 3 min), CCRF-CEM were re-suspended with PBS (1 ml) for flow cytometry measurements. Samples were analyzed on a flow cytometer (BD LSR II, BD Biosciences). Post analysis processing was carried out in FlowJo (BD).

Inhibiting Complement Mediated Lysis with Coating of PEG-Phospholipid

In order to investigate if the PEG-phospholipid was able to protect against complement mediated lysis, RBCs were used from 13 different donors who were typed for Rh complex and ABO antigens. RBCs were isolated and preincubated with PEG-phospholipid at varying concentrations, free PEG or PBS as described above. Cells were then washed (and the supernatant stored for analysis) with PBS (800 g×5 min, RT) and incubated with human serum (where the complement system is active) for 20 minutes at 37° C. They were then spun down again (800 g×5 min) and the supernatant was collected for analysis of hemolysis. The RBCs were analyzed for complement fixation (cells were incubated for 10 min with a

FITC-labelled anti-C3c antibody). RBCs were resuspended with PBS (1 mL) for flow cytometry measurements. All samples were analyzed on an Accuri C6 flow cytometer, additional controls in the form of isotype control and unstained control were ran in parallel. Post analysis processing was carried out in FlowJo (BD). The supernatant was analyzed on a variable wavelength detection plate reader, Synergy HTX, for the absorbance at 540 nm. RBCs, which were lysed via osmotic stress, were run in parallel as positive controls. The background absorbance at 540 nm from PBS containing no cells was subtracted and the difference in absorbance between cells coated with PEG-phospholipid or PBS was then calculated.

### Results

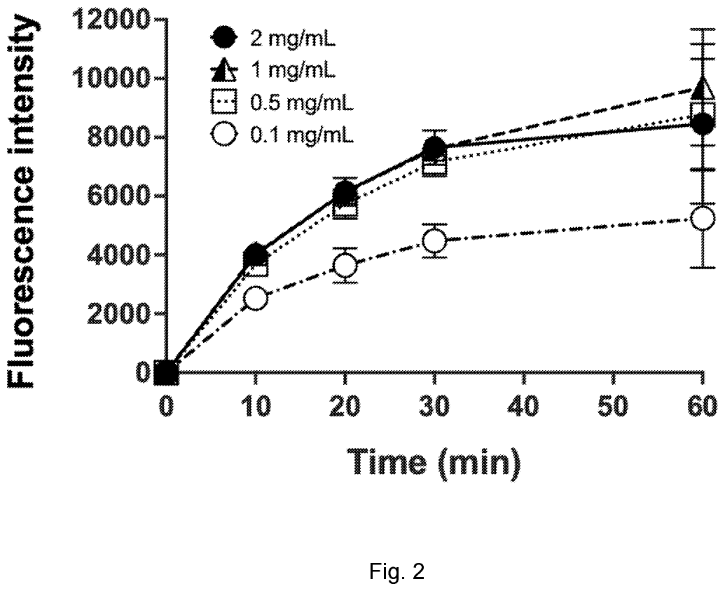

Cell surface modification was studied with FITC-labelled PEG-phospholipid and CCRF-CEM cells (5×10⁶). The cells were incubated with varying concentrations of FITC-labelled PEG-phospholipid (0.1, 0.5, 1.0, and 2.0 mg/ml in PBS) or PBS without PEG-phospholipid (as control). Cells were incubated at room temperature for 10, 20, 30, or 60 minutes with gentle mixing. After removing excess FITC-labelled PEG-phospholipid, the remaining PEG-phospholipid on the cell surfaces was quantified with flow cytometry. The binding of FITC-labelled PEG-phospholipid onto the cell surface was dependent on the incubation time and the concentration, see FIG. 2. The binding of FITC-labelled PEG-phospholipid reached a plateau after 30 minutes of incubation. Besides, no differences in the binding of FITC-labelled PEG-phospholipid were observed at concentrations above 0.5 mg/ml. In conclusion, these results demonstrate a rapid coating of the cellular membrane with FITC-labelled PEG-phospholipid and show that the reaction is saturable.

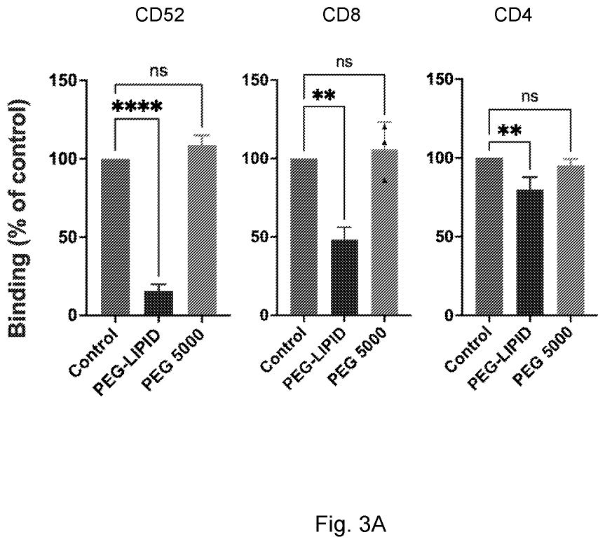

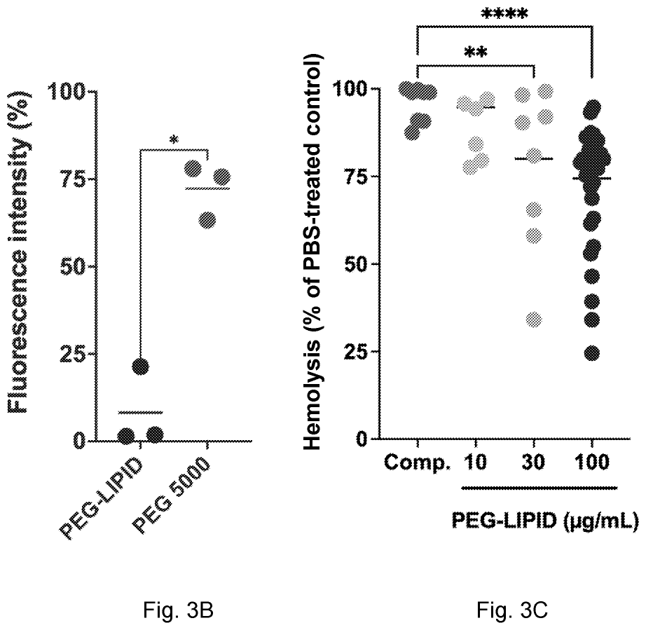

CCRF-CEM cells were incubated with PEG-phospholipid (2 mg/ml in PBS), free PEG (5 kDa, equivalent molecular weight used in the synthesis of PEG-phospholipid) or PBS (vehicle control) for 30 minutes at room temperature. After removing excess PEG-phospholipid, FITC-labelled antibodies directed towards surface antigens of different sizes, CD52, CD4, and CD8, were added. Samples were analyzed on a flow cytometer. Antibodies directed towards CD52, a small surface antigen (around 1 nm), were entirely blocked by PEG-phospholipid (FIG. 3A). The binding capacity of CD8-antibodies was reduced but not entirely blocked by PEG-phospholipid (FIG. 3A). The size of CD8 is estimated to be around 5-10 nm based on the crystal structure of the non-membranous part. Lastly, CD4-antibodies were not blocked by PEG-phospholipid, as expected, given that the size of the antigen (15 nm) is larger than the size of PEG-phospholipid (estimated to be around 6 nm) (FIG. 3A). Free PEG did not significantly influence the binding capacity of antibodies towards any of the surface antigens.

Blood group antigens are important surface antigens within the field of transplantation. Flow cytometry was used to assess the binding ability of fluorescence-labelled anti-blood group B antigen-antibody after coating with PEG-phospholipid. Erythrocytes were isolated from healthy human donors with blood type B, the red blood cells (RBCs) were incubated either with PEG-phospholipid (2 mg/ml in PBS) or PBS (control) for 30 min at room temperature. The binding capacity was effectively reduced by PEG-phospholipid-coating on RBCs were assessed by incubating coated RBCs with plasma containing complement factors and anti-blood group antigen B antibodies at 37° C. for 20 min, see FIG. 3B. Complement fixation (using an anti-C3c antibody) and lysis was studied using flow cytometry. The PEG-phospholipid reduced complement-mediated RBC lysis in a dose dependent manner in ABO missmatched donors compared with non-treated controls. (FIG. 3C).

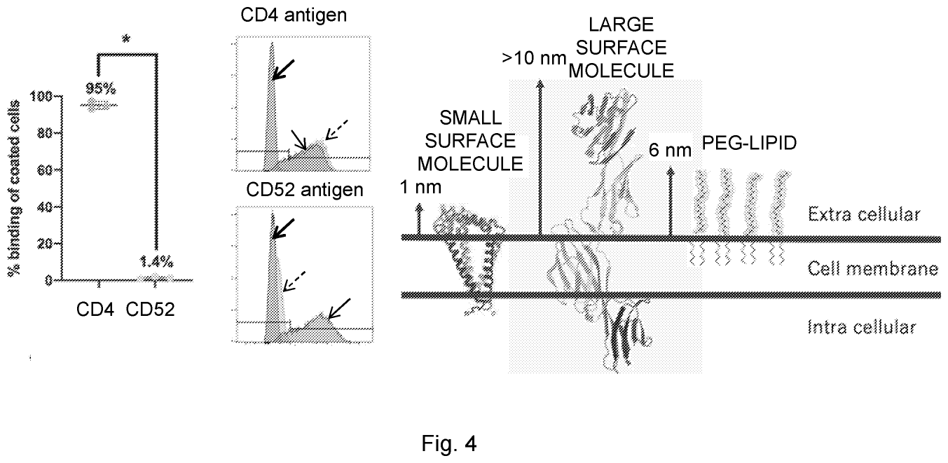

PEG is a linear polymeric substance, and the molecular mass correlates to the length of the polymer, leading to the protrusion from the cellular membrane. PEG in PEG-phospholipid has a mass of 5 kDa, which is approximately 6 nm in size, leading to a 6 nm PEG layer when anchored in the membrane. The size of blood group B antigen, CD52, CD8 and CD4 from the cell surface is approximately 1 nm, 1 nm, 5-10 nm and 15 nm, respectively. Therefore, this is in accordance with a size dependent inhibition. A clearer inhibition of the antibody binding was seen to CD8 than to CD4. The stalk like region of CD8 makes the molecule less protrusive from the membrane even though CD8 and CD4 are of similar size, see FIG. 4.

### Example 2

This Example investigated the effect of PEG-phospholipid in masking Rhesus factors D, C, c, E and e on erythrocytes.

### Materials and Methods

Whole blood samples (one O, Rh+ blood sample and two A Rh+ blood samples) from the blood center were centrifuged in Eppendorf tubes in an Eppendorf 5810R centrifuge at 2500 rpm for 10 min at room temperature (20-25° C.) and the plasma was removed and discarded. The cell fractions were subject to three washing steps in 50 ml falcon tubes. Each washing step involved adding 45 ml phosphate buffered saline (PBS) to 1 ml cell fraction followed by centrifugation at 2500 rpm for 10 minutes at room temperature.

100 μl cell suspension was mixed with 900 μl PBS and 1000 μl PEG-phospholipid (4 mg/ml, polyethylene glycol-phospholipid with PEG of 5 kDa (PEG-phospholipid) in PBS, SUNBRIGHT® PP-050CN, N-(methylpolyoxyethylene oxycarbonyl)-1,2-dipalmitoyl-sn-glycero-3-phosphoethanolamine, sodium salt, NOF Corp., Japan) to coat the erythrocytes with 2 mg/ml PEG-phospholipid (final concentration) or 1000 μl PBS as control. The erythrocyte samples were incubated 30 min at 37° C. in an incubator under rotation in a POL-EKO laboratory incubator. The erythrocyte samples were washed three times with 2 ml PBS and centrifuged at 2500 rpm for 5 min at room temperature in a Heraeus Biofuge Pico centrifuge.

The washed erythrocyte samples were then subject to an agglutination test and Rh phenotyping. In more detail, EDTA-blood was drawn and RBCs were collected and washed in PBS (1 mL blood in 45 mL PBS) three times. Then, 100 UL aliquots of RBCs were incubated with 1.9 mL PEG-phospholipid in PBS or with PBS only (control) at 37° C. for 30 min in a rotating incubator. The RBCs were washed three times with PBS (2 mL) and suspended to 50% hematocrit. Thereafter the treated and control RBCs were subjected to blood group testing using Diaclon ABO/Rh IDcards (Biorad) according to the manufacturer's instructions.

### Results

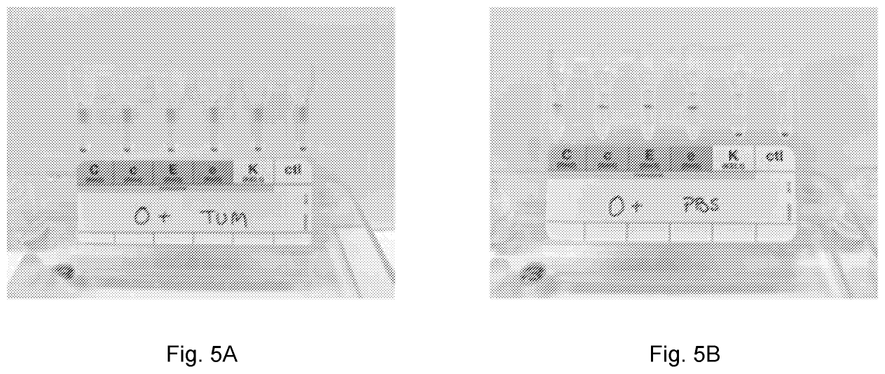

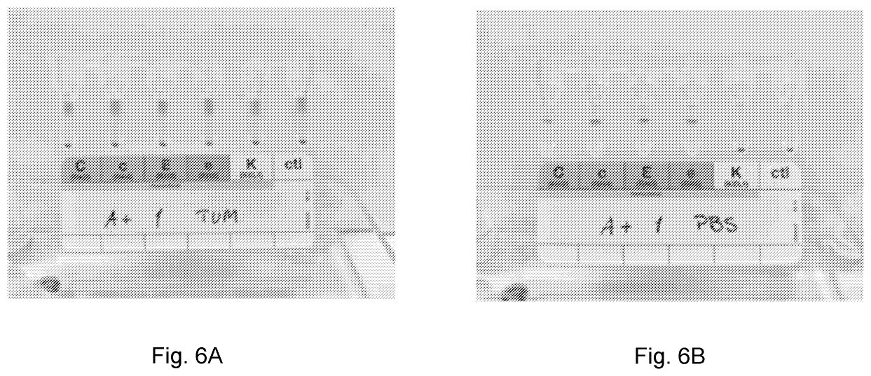

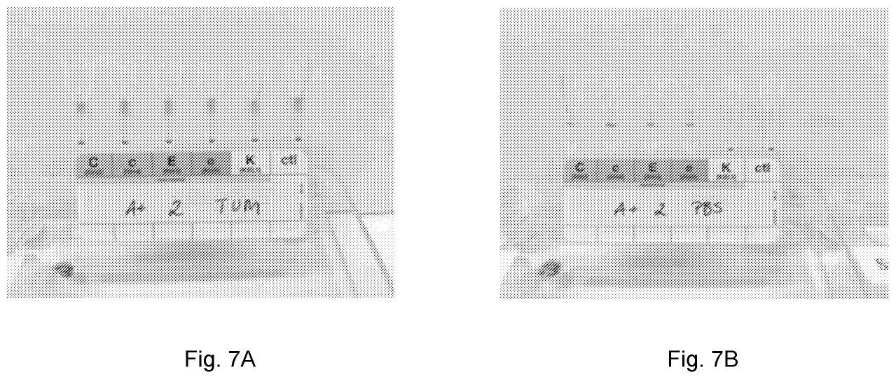

The results from the agglutination tests are presented in FIGS. 5A to 7B for the 0 Rh+ blood sample (FIGS. 5A and 5B), and the two A Rh+ blood samples (FIGS. 6A and 6B, 7A and 7B). As is clearly seen in FIGS. 5B, 6B and 7B, the untreated control led to agglutination. However, treating the erythrocytes with PEG-phospholipid masked the Rhesus factor antigens on the cell surfaces and thereby inhibited agglutination (FIGS. 5A, 6A and 7A).

Hence, the PEG-phospholipid was able to mask Rhesus factor antigens on the cell surface of erythrocytes and was thereby able to prevent agglutination of erythrocytes.

### Example 3

This Example investigated the effect of PEG-phospholipid in masking cell membrane proteins on human umbilical vein endothelial (HUVEC) cells.

### Materials and Methods

### Preparation of HUVECs

HUVEC cells were cultured with endothelial cell culture medium with supplements (Promocell) in cell culture flasks (Thermofisher Scientific) and Petri dishes (NUNC). The cells were detached using trypsin EDTA buffer for flowcytometry analysis (BD accuri C6 Serial no. 5173). The cells were rinsed with PBS (10 mL) by centrifugation (120×g, 4° C., 3 min) once and fresh medium was added to the detached cells.

Masking of Antigens on HUVECs by Coating with PEG-Phospholipid

One hundred μL of HUVEC cell pellet (5×10⁴ cells) were incubated in 0.5 mg/ml of PEG-phospholipid solution (final concentration, polyethylene glycol-phospholipid with PEG of 5 kDa (PEG-phospholipid) in PBS, SUNBRIGHT® PP-050CN, N-(methylpolyoxyethylene oxycarbonyl)-1,2-dipalmitoyl-sn-glycero-3-phosphoethanolamine, sodium salt, NOF Corp., Japan) or PBS for 45 mins at RT. Then, the cells were incubated with 5 μg/mL of FITC labelled antibodies for 15 min at RT: CD31 antibody (5 μg/ml) or isotype control Ab-FITC (BD biosciences) or with rabbit anti von Willebrand Factor (vWF) with rabbit IgG as control, both visualized by Alexa 488 goat anti-rabbit antibodies as given in Table 1.

In addition, HUVEC cells were incubated with PEG-phospholipid (0.5 mg/ml), with 10 mM free PEG (5 kDa), or PBS (control) at RT for 45 min followed by washing with PBS, and incubation in human serum with EDTA-plasma as control, both diluted 1:50 in PBS, for 30 min at 37° C. After washing, lectin pathway recognition molecules collectin-11 (CL-11), and mannan binding lectin (MBL), were detected using the FITC-labelled and isotype control antibodies as given in Table 1. For both analytes, value are presented after subtraction of the levels of binding in EDTA plasma. In addition, FITC-labelled concavalin A (a lectin of broad specificity) was incubated with coated and native HUVECs (without prior exposure to plasma) at a concentration of 50 g/ml.

After incubation with detecting antibodies, the cells were washed with PBS. All samples were analyzed on an Accuri C6 flow cytometer, unstained cells were used to gate the endothelial cell population. 10,000 events were recorded and additional controls in the form of isotype control and unstained control were ran in parallel. Post analysis processing was carried out in FlowJo.

TABLE 1
Antibodies

| | | | |
|---|---|---|---|
| Antigen | ≈size | Manufacturer, Ab specificity | Ab clone |

HUVECs

| | | | |
|---|---|---|---|
| CD31 | 15 nm | BD, mAb FITC α-CD31 | WM-59 |
| vWF | >30 mm | Biorbyt, rabbit pAb α-vWF | orb158717 |
| Rabbit | | Abcam Alexa 488 goat α-rabbit | ab 150077 |
| secondary | | | |

HUVECs plasma treated

| | | |
|---|---|---|
| CL-11 | Mybiosource, rabbit pAb | COLEC-11 |
| | α-CL-11 | |
| MBL | Hycult, mAb α-MBL | 3E7 |

### Results

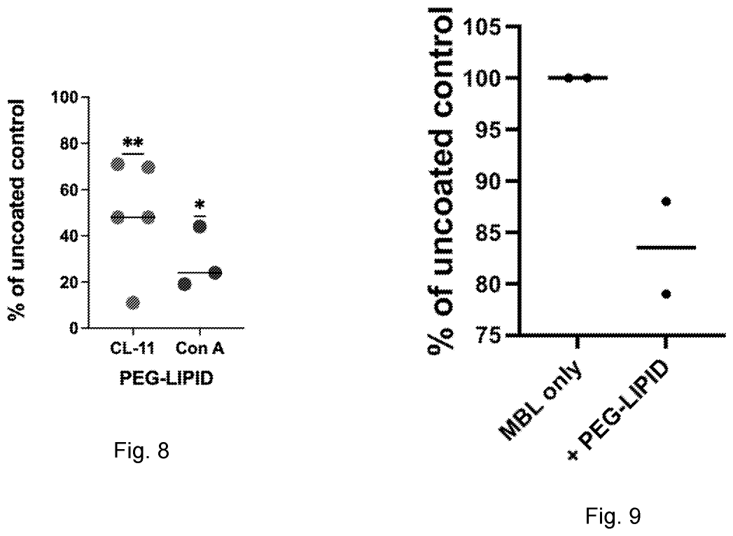

PEG-phospholipid treated HUVEC cells were incubated with human plasma, followed by assessing the binding of FITC-labeled antibodies directed against CL-11 or with the lectin Concanavalin A, without plasma exposure. In both cases, the binding was substantially reduced by PED-lipid (FIG. 8).

PEG-phospholipid coating induced a modest masking of CD31 (<20%), did not decrease the detection of vWF, and caused a marked decrease (25-30%) of MBL binding from human serum, see FIG. 9.

The mechanism by which ischemia reperfusion injury (IRI) is induced is not yet established, but binding of MBL, CL-11, MASP-2 and natural IgM antibodies have been suggested to bind to ligands on the ischemic cells thereby triggering the IRI. We hypothesize that the PEG-phospholipid coating shields off the endothelial and tubular cell membranes by hindering cell membrane proteins to reach out and bind plasma proteins in the blood in the kidney graft after reperfusion. Our data corroborates this concept in that the PEG-phospholipid effectively shields off small surface antigens such as CD52 and the Rh antigens, see Examples 1 and 2, which are extending approximately 1 nm from the surface. Also, CD8, which is ranging between 5-10 nm based on the crystal structure of the non-membranous part, is partially blocked by the PEG-phospholipid coat, while the PEG-phospholipid layer did not reach out to cover taller antigens such as CD4 with the estimated molecular extension of 15 nm from the membrane, see Example 1. Also antibodies to CD31 and vWF (extending 15 nm and 30 nm, respectively, from the surface) were not affected. These results are compatible with that PEG-phospholipid has a 5 kDa PEG chain with a theoretical 6 nm extension out from the cell membrane (FIG. 4).

### Example 4

This Example investigated pharmacokinetics of PEG-phospholipids in pig kidneys in vivo.

### Materials & Methods

### Preparation of PEG-Phospholipid Derivatives

PEG-phospholipid was purchased from NOF corporation, Tokyo, Japan (SUNBRIGHT® PP-050CN, N-(methylpolyoxyethylene oxycarbonyl)-1,2-dipalmitoyl-sn-glycero-3-phosphoethanolamine, sodium salt). Fluorescein-labelling was performed by conjugating 5FAM-GC-OH (5-carboxy fluorescein) to Mal-PEG-phospholipid. First, Mal-PEG (5 kDa)-DPPE was synthesized by combining a-N-hydroxysuccinimidyl-w-maleimidyl poly(ethylene glycol) (180 mg, NHS-PEG-Mal, Mw: 5 kDa, NOF), triethylamine (50 μL), and DPPE (26 mg) with dichloromethane and stirring for 36 h at room temperature (RT). Precipitation with diethyl ether yielded Mal-PEG-phospholipid as a white powder (190 mg, 95% yield). 5FAM-GC-OH (FAM-GC) was conjugated to Mal-PEG-phospholipid. Briefly, 107 μL FAM-GC (10 mg/mL) in DMSO was mixed with 1 mL Mal-PEG-phospholipid (10 mg/mL in PBS) for 24 h at RT. The fluorescein-GC-conjugated PEG-phospholipid (FAM-PEG-phospholipid) was purified using spin columns before applying for cell modification (Sephadex G-25, GE Healthcare, Buckinghamshire, UK). The resulting labelling was approximately 0.7 mol FAM/mol PEG-phospholipid.

Biotin-PEG-DPPE (Biotin-PEG-phospholipid) was synthesized by combining poly(ethylene glycol) (N-hydroxysuccinimide 5-pentanoate) ether 2-(biotinylamino) ethane (biotin-PEG-NHS, Mw: 5000 Da from NOF, 180 mg) and DPPE (20 mg) with triethylamine (50 μL) and dichloromethane (4 mL) and stirring for 48 h at RT 2. Precipitation with diethyl ether yielded biotin-PEG-lipid as a white powder (165 mg; 80% yield). All preparations of PEG-phospholipid were used at a final concentration of 2 mg/mL.

### Culture of CCRF-CEM Cells

The CCRF-CEM cell line was applied in this experimental setting due to its floating characteristics and, therefore, the use of trypsin during the splitting process could be avoided, which is beneficial as trypsin may affect the PEG-phospholipid coating of the cell surface. The CCRF-CEM cell line was purchased from the Health Science Research Resources Bank (Tokyo, Japan). CCRF-CEM cells were cultured (at 37° C., 5% CO₂) in RPMI-1640 medium (containing 10% heat inactivated fetal bovine serum, Penicillin 50 IU/mL, and Streptomycin 50 μg/mL).

Retention Time, Cell Growth and Viability after PEG-Phospholipid Modification

CCRF-CEM cells (3×10⁶ cells) were collected and rinsed with PBS (3 mL) by centrifugation (120×g, 4° C., 3 min) once. The cell pellet was suspended in FAM-PEG-phospholipid (50 μl, 2 mg/ml in PBS) or PBS (50 UL, as control) for 30 min at RT with gentle agitation. The cells were rinsed with PBS (10 mL) by centrifugation (120×g, 4° C., 3 min) once, and the cell pellet was suspended in RPMI 1640 (1 mL). The coated cells were seeded in a 6-well plate (5×10⁵ cells/mL in 5 mL RPMI 1640) and the viability, and cell number were calculated using a cell counter (dead cells were stained with trypan blue) at 0, 1, 2, 4 and 7 days of incubation. Binding of FAM-PEG-phospholipid bound to the cell surface were assessed by Confocal laser scanning microscopy and flow cytometry at the same time points.

### Vascular and Tubular Distribution and Detachment of PEG-Phospholipid In Vivo

Full information on donor and recipient pigs is given in Example 5. Donor kidneys were treated with 10-15 mL biotin-PEG-phospholipid solution with a concentration of 2 mg/mL. Four recipient pigs were transplanted with biotin-PEG-phospholipid-treated kidneys. Their native kidneys were not removed and remained in the animals throughout the study. The four recipient pigs were euthanized after 12, 24, 48 and 72 h, respectively and biopsies were taken at euthanasia from the biotin-PEG-phospholipid treated kidney and one of the native kidneys. Frozen sections (4 μm thickness) were incubated in Alexa488-streptavidin (GE Healthcare, 1:500) at RT for 10 min. The sections were analyzed by laser-scanning confocal microscopy (LSM510, META, Carl Zeiss, Germany). Slides were visualized using a Zeiss Axio Imager A1 microscope.

### Results

### Dose-Response Binding of PEG-Phospholipid to Cell Surfaces

In order to optimize the PEG-phospholipid binding to cells, a non-adherent cell line (CCRF-CEM) was used. CCRF-CEM cells were incubated with FAM-PEG-phospholipid (up to 2.0 mg/mL), and the amount of bound PEG-phospholipid was determined by flow cytometry. The binding of PEG-phospholipid to the cell surface was dependent on both the incubation time and concentration (FIG. 2). The binding of PEG-phospholipid at all concentrations reached a plateau after 30 min and no clear increase in binding of FAM-PEG-phospholipid was observed at concentrations ≥0.5 mg/ml FAM-PEG-phospholipid, corroborating a rapid and saturable coating of the cell membranes.

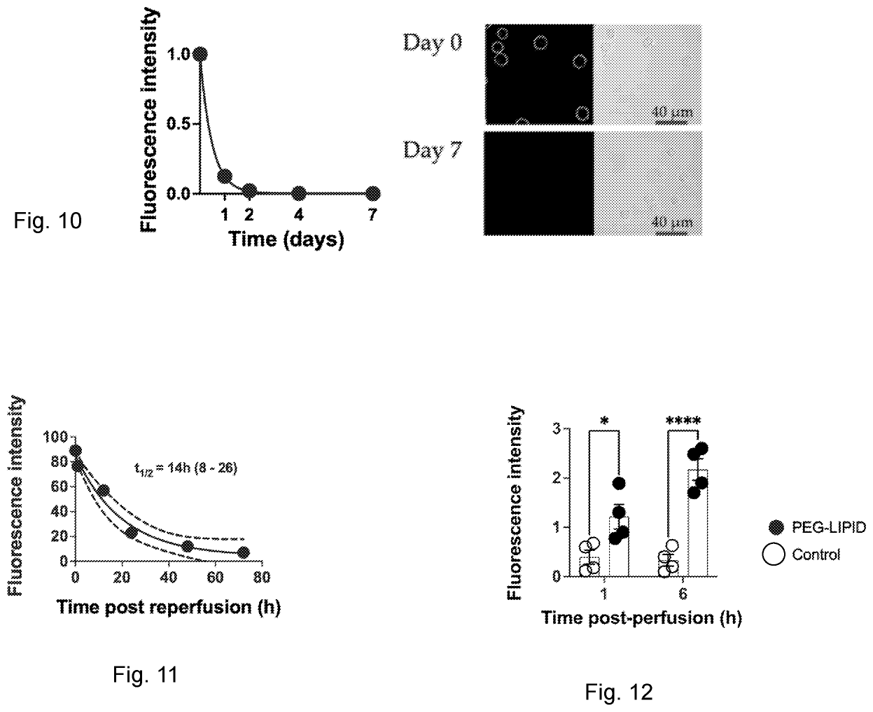

Detachment and half-life of PEG-phospholipid in vitro and in vivo Detachment of FAM-PEG-phospholipid from CCRF-CEM cells in vitro was studied by flow cytometry and confocal microscopy over time (FIG. 10). The fluorescence signal was minimal after 2 days and no longer detected after 4 days in culture. The PEG-phospholipid binding was restricted to the cell membrane and not taken up by the cells.

The kinetics of PEG-phospholipid attached to the kidney parenchyma in vivo was studied in a porcine survival model (see Example 5), where pigs were transplanted with grafts treated with biotin-PEG-phospholipid ex vivo. The animals were sequentially euthanized. From the fluorescence analysis by confocal microscopy with Alexa488-streptavidin, the half-life of PEG-phospholipid attached to the kidney was calculated. A one-phase exponential decay model was applied to fit a curve to the data points and the half-life of PEG-phospholipid bound to the kidney parenchyma was calculated to be approximately 14 h (SD: 8-26) (FIG. 11). The autologous kidneys of the recipient were also preserved and analyzed in order to estimate the systemic leakage of PEG-phospholipid, which was shown to be very low, reliably detected only after 1 hour. By contrast the FAM-PEG-phospholipid was consistently found in the urine in the acute non-survival model (ANSM, see Example 5) (FIG. 12).

From monitoring of the fluorescence of the PEG-phospholipid bound to cells in vitro, it was concluded that there was a decrease in the intensity during the observation period, indicating that PEG-phospholipid detached from the endothelium. In vitro the PEG-phospholipid detached fully after 48 h. In the ANSM in vivo, it was demonstrated that the fluorescence seemed to migrate from the glomeruli to the tubular system within 6 hours post-transplantation. These data were supported by the fact that FAM-PEG-phospholipid fluorescence was detected up to 12-24 h after reperfusion in a survival study, which was completely lost after 48 hours. A t12 of 14 h was calculated, supporting that the PEG-phospholipid construct was excreted via the kidneys, the fluorescence was found in the urine during the observation period of the non-survival study.

### Example 5

This Example tested PEG-phospholipids in three different porcine transplantation models, an acute non-survival model (ANSM), an allogeneic survival model (SM) and an clinical-like allogeneic survival model (CLSM), without immunosuppression, up to four days.

### Materials & Methods

### Preparation of PEG-Phospholipid Derivatives

PEG-phospholipids were prepared as described in Example 4.

### Routine Clinical Chemistry Markers

Before surgery for both long term survival studies (SM and CLSM), samples were collected in EDTA tubes from both donors and recipients for analyses of total and differential white blood cell counts and hematology (EPK, Hb, EVF, MCV, MCHC, reticulocytes, thrombocytes). Plasma samples were analyzed for aspartate amino transferase (ASAT), alanine aminotransferase (ALAT), γ-glutamyltransferase (GT), glutamate dehydrogenase (GLDH), and creatinine at the Section of Clinical Chemistry, SLU, Uppsala.

During and after surgery, systemic blood was collected for up to 4 days and analyzed for creatinine, sodium, potassium and phosphate (Section for Clinical Chemistry, Uppsala University Hospital).

### Complement and Coagulation Activation Markers in EDTA-Plasma

Commercially available kits were used to assess the levels of the complement activation products C3a as well as soluble C5b-9 (sC5b-9) in porcine plasma according to manufacturer's instructions (ABIN2543272 and ABIN6202276; Antibodies-online.com). Human C3a was measured using anti-human C3a mAb 4SD17.1 for capture and biotinylated polyclonal rabbit anti-C3a antibody for detection, and human sC5b-9 using mAb anti-human neo-C9 aEII (Bioporto Diagnostics A/S, Hellerup, Denmark) for capture and polyclonal sheep biotinylated anti-Hu-C5 antibody (BP373, OriGene, Herford, Germany) for detection.

TAT was measured in both human and porcine plasma using an anti-human thrombin monoclonal antibody was used for capturing and an HRP-coupled anti-human antithrombin (AT) antibody was used for detection (Enzyme Research Laboratories, South Bend, IN, USA). FXIla-C1INH complexes were analyzed by a sandwich ELISA, essentially as described in 6 using goat anti-human FXII polyclonal antibodies (Enzyme Laboratories) for capture and goat anti-C1INH antibodies (Enzyme Reseach Laboratories) for detection. It should be noted that the assays used here for human and porcine TAT and FXIIa-C1INH complexes are fully cross-reactive while those for C3a and sC5b-9 are not.

### Cytokines and Chemokines

Multiplex protein analyses using multiplex Luminex xMAP Technology (Millipore Corporation, Billerica, MA, USA) were performed using a porcine specific kit for 12 cytokines/chemokines: Interferon-gamma (INFγ), Interleukins IL-1b, IL-2, IL-1a, IL-1RA, IL-4, IL-6, IL-10, IL-18, IL-8, IL-12, and TNF. Plasma was incubated with agent-specific-coloured magnetic beads, thereafter with detection antibodies and streptavidin-phycoerythrin (Millipore Corporation). Plates were measured using a MAGPIX instrument (Luminex Corporation, Austin, TX, USA). Raw data [median fluorescence intensity (MFI)] was translated into protein concentration (ng/ml) using a standard curve.

In addition, TNF, IL-1b, IL-6 and tissue factor (TF) were quantified in plasma by immunoassays using a GyroLab workstation (Gyros, Uppsala, Sweden), and the presence of these compounds, as well as IFNγ in biopsies (below) were verified by PCR.

### Kidney Biopsies

### Detection of PEG-Phospholipid by Histofluorescence

Frozen sections (4 μm thickness) of kidneys treated with biotin-PEG-phospholipid and control kidneys were incubated in Alexa488-streptavidin (GE Healthcare, 1:500) at RT for 10 min. The sections were analyzed by laser-scanning confocal microscopy (LSM510, META, Carl Zeiss, Germany).

### Immunohistochemistry

Paraffin sections of porcine kidney tissue were cut (3-5 μm), deparaffinized in xylene and rehydrated with a graded series of ethanol and distilled water. For IHC staining a heat-induced antigen retrieval was performed by boiling the sections in sodium citric buffer (pH 6), the sections were blocked with 10% normal goat serum (Jackson Immunoresearch) before primary antibody incubation: anti-MAC (abcam ab66768); anti-C3b alpha chain (Bioss bs-4873R); anti-C4d (abcam ab64157), anti-C5aR (Acris AP06509PU-N), anti-HO1 (abcam ab52947), anti-CSE (abnova H00001491-M01), anti-Nitrotyrosine (Merck Millipore AB5411), anti-NOS2 (Santa Cruz sc-651), detection was performed by DAKO Real

Detection System alkaline phosphatase red (Dako). The sections were counterstained with Mayer's hematoxylin (Sigma #51275). Control staining was done with a nonspecific rabbit IgG (Dako #X0936). The slides were visualized using a Zeiss Axio Imager A1 microscope with a ×10 objective (100-fold magnification). Intensity of staining was quantified on multiple randomly selected 800,000 μm² sections using the AxioVision 4.8 software (Zeiss). Data are presented as mean densitometric sum red.

Quantitative RT-PCR for cytokines in tissue samples Snap frozen kidney wedge biopsies were shredded in a QIA shredder (Qiagen, Uppsala, Sweden) and stored in −70° C. Total RNA extraction and cDNA synthesis were performed utilizing RNAeasy Mini kit (Qiagen, Holden, Germany) and Superscript II Reverse Transcriptase kit (Invitrogen, Stockholm, Sweden), respectively. All cDNA sequences of porcine genes were obtained from Genbank. All primers were provided by Invitrogen. Gene expression was determined by RT-PCR. Specificity of PCR products was assessed by electrophoresis performed on 2% agarose gel. mRNA expression was normalized to beta-actin and quantitative values were obtained from the threshold cycle number (Ct) and the fold change in expression was evaluated using the ΔΔCt method. The selected analytes included the proinflammatory cytokines IL-1b, IL-6, IFNγ and TNF, as well as TF.

### NETs Analysis

Deparaffinized renal tissue samples were fixed for 30 min at room temperature using 2.5% glutaraldehyde in 0.15 mol/L sodium cacodylate, pH 7.4 cacodylate buffer, and used for detection of NETs. After fixation, the samples were washed with cacodylate buffer and dehydrated with an ascending ethanol series (10 min/step) from 50% (vol/vol) to absolute ethanol. Specimens were subjected to critical-point dying in carbon dioxide, with absolute ethanol as the intermediate solvent. The specimens were examined using a HITACHI SU3500 scanning electron microscope at the Mbio-Microscopy Facility at Department of Biology. The location of individual target molecules was analyzed at high resolution by ultrathin sectioning and transmission immunoelectron microscopy. The coverslips with specimens were embedded in Epon 812 and sectioned with a diamond knife in an ultramicrotome into 50-nm-thick ultrathin sections. For quantification of NET areas, characteristic web-like fibrillar structures were first identified at high magnification. Quantitative NETs surface area assessment was performed using Adobe Photoshop CS5. In short, the number of pixels/square micrometer was determined by using the Ruler Tool. NET areas were then translated into pixel numbers with the Magic Wand Selection Tool. Thus, the fractions of the NET area relative to the entire area of a given electron micrograph was calculated.

### Urine

In the acute non-survival model, the ureters from both kidneys were separately catheterized and serial urine samples were collected for 6 hours. The amount of fluorescein-labelled FAM-PEG-phospholipid in the urine was quantified in samples added into black microwell plates (Thermo scientific, 96F Maxisorb; Black microwell plates; 43711) and measured by using the fluorescence plate reader Synergy HTX with excitation wavelength of 485 nm and emission wavelength of 528 nm.

### Experimental Design—In Vivo Studies on Pigs

The pig was chosen as a large animal model for allogeneic transplantation, because of its anatomy and physiology, which closely resemble that of humans. Specifically, the structure and function of the kidney is very similar to the primate organ. The porcine kidney is described as true multirenaculate, multipapillate with a calyceal system like that of humans. Blood supply divides transversely between the cranial and caudal poles, rather than longitudinally like in most other species. Pigs have a nephron type similar to that of humans and renal cytochrome enzymes (P450 3A, 2A, 2C) exhibit activities similar to their human homologous enzymes. Further, there are many examples of similar or identical genes that encode for enzymes present in the kidneys of both humans and pigs, e.g., fucosidase, galactosidase, prostaglandin 9-ketoreductase and dipeptidyl-aminopeptidase IV. In addition, there are considerable similarities between the human and porcine immune systems with >80% resemblance in studied parameters. The corresponding resemblance between human and parameters is only approximately 10%.

### Three Porcine Allogeneic Transplantation Models

To study the effects of the PEG-phospholipid coating in porcine allogeneic transplantation, one acute non-survival model (ANSM), one allogeneic survival model (SM) and one clinical-like allogeneic survival model (CLSM) were employed, in addition to the in vivo study to investigate tissue distribution and detachment (Example 4). Samples: plasma, biopsies and urine were collected as indicated below in the sections describing each model and analyzed as described above.

TABLE 2
Summary of the four allogeneic transplantation setups

| | | PEG- | | | |
|---|---|---|---|---|---|
| | | PHOSPHO- | Con- | | |
| | Donors | LIPID | trols | Observation | |
| Study | (n) | (n) | (n) | time | |
| Acute non-survival | 6 | 6 + 6 | | 6 | h |
| model (ANSM) | | (transplanted en bloc) | | | |
| Allogeneic survival | 5 | 6 | 4 | 96 | h |
| model (SM) | | | | | |
| Clinical-like allogeneic | 5 | 5 | 5 | 96 | h |
| survival model (CLSM) | | | | | |
| Tissue distribution/ | 2 | 4 | — | 12-72 | h |
| detachment | | | | | |

### Porcine Acute Non-Survival Transplantation Model (ANSM)

The study was approved by the Ethics Committee for Animal Experimentation, Uppsala, Sweden, Dnr C175/12.

Twelve pigs (Yorkshire×Landrace×Hampshire) aged 10-12 weeks, and weighing 30-35 kg, were purchased from a conventional farm with high health pigs. The farm is under supervision by swine specialists from the Veterinary Faculty at SLU. The animals were transported to the animal experimental facility at Uppsala University Hospital where they were immediately subjected to a non-survival transplantation procedure.

### Anesthesia

Upon arrival to the facility, an intramuscular (IM) injection of 2.2 mg/kg xylazine (Rompun®, Bayer, Leverkusen, Germany) in combination with 6 mg/kg zolazepam-tiletamine (Zoletil 100@, Virbac, Carros, France) were given for induction. Subsequently, the animals were secured in a prone position. Peripheral venous catheters were introduced in both ears for TIVA anaesthesia; midazolam 0.105 mg/kg/h (Midazolam Actavis 5 mg/mL, Actavis AB, Sweden), ketamine 28 mg/kg/h (Ketaminol® vet, 100 mg/ml Intervet AB, Sweden), fentanyl 3.5 μg/kg/h (Fentanyl B. Braun 50 μg/mL, B. Braun Medical AB, Sweden) and fluid administration of lactated Ringer solution (Ringer-acetate, Fresenius Kabi AB, Sweden) approximately 10 ml/kg/hour and succinylated gelatine (Gelofusine®-B. 40 mg/mL, Braun Melsungen

AG, Germany). An epitracheal mini-incision was made for exposure of trachea and transtracheal intubation. The animals were mechanically ventilated with 30% oxygen in air to obtain 5.0-5.5 kPa CO₂ (Servo-I Mechanical Ventilator, MAQUET, Medical Systems, US.) A mean arterial pressure (MAP) of >60 mm Hg was aimed to ensure sufficient organ perfusion.

### Transplantation Surgery

We developed a porcine transplant model enabling a dual en bloc retrieval and implantation of both kidneys from a donor into a recipient animal. By isolated ex vivo PEG-phospholipid incubation of just one randomly selected kidney within the en bloc package, this technique allowed us to co-transplant the treated organ with its perfectly matched control organ into one recipient pig, which not only reduces the number of experimental pigs but also minimizes experimental confounding variables.

Both kidneys and the trunk of the supra- and infrarenal aorta as well as the inferior vena cava were mobilized to create an en bloc package consisting of the two kidneys with corresponding ureters and the renal vasculature. The en bloc package was immediately removed and cold flushed ex situ on the back table with cold HTK solution. Here, the time between in situ clamping in the donor and ex situ perfusion with cold HTK solution was less than 5 minutes causing an initial warm ischemia in this model. Thereafter, the en bloc package was cold stored in HTK solution at 4° C. for 24 hours. Post-retrieval, the donor pigs were euthanized by an IV overdose of pentobarbital sodium (Euthasol® vet. 400 mg/mL, Virbac).

Post-preservation, one kidney within each en bloc package was randomly selected for PEG-phospholipid incubation. The vein and the artery of the contralateral kidney (control) as well as the vein of the selected kidney were clamped. A total of 3-5 mL of PEG-phospholipid solution with a concentration of 2 mg/ml was slowly infused in the selected kidney. The artery of the treated kidney was then clamped and the en bloc package was cold stored for another 40 min at 4° C. in HTK. Post-incubation and before the implantation, the redundant PEG-phospholipid was flushed from the treated-kidney with HTK without removing the clamps from the control.

The recipient pigs were handled and anaesthetized as previously described. The en bloc package was placed horizontally intra-abdominally. The distal end of vena cava and aorta of the transplant was anastomosed end-to-side to the recipient's cava and aorta, respectively. Upon de-clamping, the treated and control kidney were sequentially reperfused (by delayed declamping of the control kidney), while the effluent from the treated kidney was flushed out. This was done in order to prevent contamination of the control kidney from the remaining PEG-phospholipid solution within the aortic conduit. Both transplant ureters were separately catheterized for documentation of urinary output.

### Sampling

EDTA-blood was collected from both allograft veins at 1, 5, 15, 30, 60, 120, 240, and 360 min post-reperfusion. Two wedge biopsies were taken from each transplant at 1, 60, and 360 min post-reperfusion. One biopsy was snap-frozen in liquid nitrogen for cytokine analysis and PEG-phospholipid determination and the other stored in 4% PFA and used for immunohistochemical studies. The total amount of urine was collected at 360 min post-reperfusion.

### Porcine Survival Transplantation Models SM and CLSM

These studies were approved by the Ethics Committee for Animal Experimentation, Uppsala, Sweden, Dnr C123/14. For each of these studies (SM and CLSM) five donor pigs and 10 recipient pigs of both sexes were used. All pigs were certified SPF pigs (Yorkshire×Hampshire) and bred at Lövsta University herd, SLU, Sweden. At arrival to SLU the pigs were 8 weeks old.

For the SM study, the pigs were selected after typing for Swine Leucocyte Antigen (SLA) class 1 in the following manner: the donor pigs were obtained from two litters and not related to any of the recipients. The recipient pigs were siblings, matched in pairs according to SLA class 1 (see below). For the CLSM study, all recipient pigs were siblings and not related to the donors, but these the pigs were not SLA-typed.

### SLA-Typing by PCR-SSP

SLA-typing was performed with the complete set of primers specific for the alleles at three SLA class I loci, SLA-1, SLA-2 and SLA-3 and three SLA class II loci, DRB1, DQB1 and DQA 8-10, respectively. Total genomic DNA was isolated from whole blood samples of 27 purebred pigs using the GenElute Mammalian Genomic DNA Miniprep Kit (Sigma, St. Louis, MO, USA), following the manufacturer's instructions. Typing PCR reactions contained 1× TopTaq™ master mix (Qiagen GmbH, Hilden, Germany), 1× CoralLoad loading buffer (Qiagen), 0.2 pmol/μl of α-actin positive control primers, 0.2 pmol/μl of allele-specific primers (Eurofins Genomics Ebersberg, Germany) and 30 ng of DNA, in a total volume of 10 μl. Typing of each pig included a negative control without DNA to check for reagent contamination, and was set up and electrophoresed in a standard 96-well format. The thermal-cycling conditions on the T Gradient thermal cycler (Biometra, Goettingen, Germany) consisted of an initial incubation of 95° C. for 2 min, followed by 30 cycles of 95° C. for 15 s, 65° C. for 20 s and 72° C. for 20 s. PCR products were electrophoresed in 2.5% DNA grade agarose gels (Biozym Biotech Trading GmbH, Vienna, Austria) in 1×TAE buffer at 150 V for 5 min using the Micro SSP Gel System (One Lambda, Canoga Park, CA, USA) and visualized after staining with GelStar™ (Lonza, Rockland, ME, USA). Interpretation of the results was based on the presence of allele-specific PCR products of the expected size in each lane.

### Acclimatization and Training

The recipients were kept in individual pens measuring approximately 2.5 m² within sight and sound of each other. The donors were kept together in one pen. A 12:12 h light/dark schedule was used and an infrared lamp (24 h) was placed in the corner of each pen. As bedding, straw and wood shavings were used. Twice daily the pigs were fed commercial pig feed (SOLO 330, Lantmännen), the amount according to SLU regimen for growing pigs. Water was provided ad libitum. The pens were cleaned twice daily. The recipients underwent a 14 day socialization and training program, where the pigs were trained to accept touching and palpation of the ears as preparation for stress-free blood sampling from the auricular vein. Further, the animals were accustomed to an ultrasound transducer dummy over the abdomen to tolerate ultrasound examination of the urinary bladder and transplanted kidney post-operatively. The pigs were also trained to step onto a spring scale, accept free-flow urine sampling and undergo a clinical examination including auscultation of the heart and lungs. After 14 days of acclimatization and training, the recipients weighed 32.4±2.2 kg (mean±SD) and were ready for the transplantation procedures. In total, the experiment lasted for three weeks.

Prior to surgery, blood samples were analyzed for blood cell counts and hematology as described above, in order to confirm that donors and recipients in both studies were healthy.

### Anaesthesia and Analgesia

Anaesthesia was induced in the pigs' home pens with tiletamine-zolazepam (Zoletil 100@ vet. Virbac, Carros, France) mixed with medetomidine (Domitor® vet 1 mg/mL) IM according to Rydén et al. Nursing and training of pigs used in renal transplantation studies, Laboratory Animals (2020) 54 (5): 469-478. Pre-operatively, buprenorphine was given at a dose of 0.01 mg/kg BW IM and bensylpenicillinprokain (Penovet® vet. 300 mg/mL, Beohringer Ingelheim, Ingelheim, Germany) at 20 mg/kg BW IM. The animal was covered with a blanket and transported to the preparation room, where oxygen saturation and pulse rate measurements were started. To prevent heat loss, socks were put on the claws and distal legs. An intravenous (IV) catheter (BD Venflon™ 20 G, 32 mm, BD Medical, Franklin Lakes, US) was placed in an auricular vein, and blood was sampled for the analysis of complete blood cell count (CBC) and enzyme activities. Endotracheal intubation was facilitated by use of a laryngoscope. Oxygen (4 L/min) was delivered by a face mask until the animal was connected to the anaesthesia circle. Anaesthesia was maintained with isoflurane (IsoFlo vet. Orion Pharma Animal Health, Sweden) in an oxygen/air mixture (FIO2 0.3) from a rebreathing circuit and all pigs were mechanically ventilated (FLOW-i Anaesthesia Delivery System, MAQUET Medical Systems, US). Epidural morphine (Morfin Epidural Meda 2 mg/mL, Meda AB, Sweden) 0.1-0.12 mg/kg was administered according to the protocol of Malavasi et al. 2006. The pigs received IV infusion of lactated Ringer solution (Ringer-acetate, Fresenius Kabi AB, Sweden) approximately 10 ml/kg/hour and succinylated gelatine (Gelofusine®-B. 40 mg/mL, Braun Melsungen AG, Germany) as a bolus of 3 mL/kg during 10 min, followed by 3 mL/kg/hour. To achieve adequate arterial blood pressure (≥60 mmHg), dobutamine (DOBUTAMIN Carino® 250 mg/50 ml, Carinopharm, Germany) was given as an infusion (5-15 μg/kg/min). During anaesthesia, circulatory and respiratory parameters were continuously monitored (AS/3 Anesthesia Monitor, Datex-Ohmeda, Finland). Arterial blood pressure was intermittently measured oscillometrically using an inflatable cuff placed around a forelimb and connected to the monitor.

To allow repeated blood sampling in the recipient pigs, a polyurethane catheter (BD Careflow™ 3 Fr 200 mm, BD Medical, US) was introduced via the auricular vein into the jugular vein using the aseptic Seldinger technique. The catheters were sutured onto the ear with monofil-coated polyamide (Supramid 2-0, B Braun Medical, Sweden) and covered with a bandage (Snøgg AS, Norway).

### Donor Surgery and Treatment of the Graft

SM study: the kidneys were retrieved en bloc using the same procedure as in the ANSM study. Also here, the kidneys were exposed to an initial warm ischemia of less than 5 minutes. Post-retrieval, the donor pigs were euthanized by an IV overdose of pentobarbital sodium (Euthasol® vet. 400 mg/mL, Virbac). Post preservation, the kidneys were randomized to receive either PEG-phospholipid (15 mL, 2 mg/mL) or HTK solution at +4° C. by injection through the renal artery after clamping of the renal vein. During a 40-60 min incubation, the renal artery and vein were maintained clamped. The SM study comprised 6 kidneys treated with PEG-phospholipid and 4 control kidneys.

CLSM study: In this study, the retrieval of the kidney resembled the standard operative procedure of human organ procurement. Each donor was randomized to either PEG-phospholipid or HTK treatment. After warm dissection and mobilization of the kidneys, the supra-iliac aorta was clamped and a 12 french aortic cannula was inserted. Immediately after the clamping of the supra-celiac aorta, the kidneys were irrigated in situ through the aortic cannula by gravity infusion with either PEG-phospholipid (250 mL, 2 mg/mL) or HTK (250 mL) solution. Blood was evacuated through an incision in the distal portion of the cava and the kidneys were cooled in situ by filling around with slush ice. Post-retrieval, the kidneys were submerged with either PEG-phospholipid or HTK solution and cold stored at +4° C. for 24 hours. In contrast to ANSM and SM studies, the kidneys were not exposed to an initial warm ischemia. The CLSM study comprised 5 PEG-phospholipid treated and 5 control kidneys (Table 2).

### Transplantation Surgery

The recipient pigs underwent allogeneic single kidney transplantation. Through a 15-20 cm abdominal midline incision, the iliac vessels in the right iliac fossa were identified and carefully mobilized. The distal segment of the vena cava as well as the iliac artery (from its emergence from the aorta) were mobilized, sealing the surrounding lymphoid tissue. The renal graft was thereafter placed in the right iliac fossa in the proximity of the iliac vessels. The renal vein and artery were trimmed, and subsequently anastomosed in an end-to-side fashion to the recipient's right iliac artery and distal vena cava using a polypropylene 7/0 running suture (PROLENE®, Ethicon, US). The ureters were implanted by extravesical ureteroneocystostomy to the top of the bladder by 6-0 polydioxanone suture (PDS®, Ethicon, US). Thereafter, bilateral nephrectomy of the native kidneys was performed. The midline incision was closed by running a 2/0 polyglactin (VICRYL®, Ethicon, US) fascia suture and skin clips. No anticoagulants were given to the recipient. After the inguinal transplantation was completed, urine was collected directly from the bladder by cystocentesis, showing that all of the transplanted kidneys had started to produce urine soon after reperfusion. By using a urine stick the sample was analyzed for proteinurea and hematuria. The mean duration of the surgery was 2.5 hours (1.75-3.25 hours).

### Post-Operative Care

Towards the end of surgery, isoflurane administration was discontinued, the fraction of inspired oxygen was increased (FiO2 1.0) and the pigs were weaned off the mechanical ventilator. The animals were placed in a cage where they could easily be monitored continuously by the staff. Oxygen supplementation was delivered by a face mask. Heart rate, respiratory rate, oxygen saturation and body temperature were monitored until the pigs regained consciousness. At the end of anaesthesia and during the remaining post-operative period, additional buprenorphine (0.03 mg/kg) was given IV after assessment of the general condition and behavior. Antibiotic treatment with Enrofloxacin (Baytril® vet. 100 mg/mL, Bayer) at a dose of 2.6 mg/kg BW IM and bensylpenicillinprokain (Penovet® vet. 300 mg/mL, Boehringer Ingelheim) at a dose of 20 mg/kg BW IM was given the day after surgery. If needed, the pigs were hand fed fruit to stimulate appetite, supported to drink water, and assisted to stand and walk post-operatively.

### Sampling SM and CLSM Studies

During and after transplantation, EDTA blood was collected from the local renal vein at 0, 15, 30 and 60 min after reperfusion, and systemic blood samples were obtained at 1, 2, 3, and 4 days after surgery.

Two wedge biopsies were taken from each transplant before reperfusion, 15 min after reperfusion, and immediately after euthanisation. One biopsy was snap-frozen in liquid nitrogen for cytokine analysis and the other stored in 4% PFA and used for immunohistochemical studies.

### Clinical Examination Including Ultrasound Evaluation of Bladder and Kidney Transplant

Clinical appearance was assessed several times each day after surgery and a thorough clinical examination conducted once daily. The urinary bladders were examined every day at 9 am and 6 μm to determine if the transplanted kidney produced urine. When possible, free-flow urine was collected and evaluated macroscopically, and density, pH, blood and protein were measured.

Each kidney was examined once after transplantation by ultrasound (Logiq e R6, GE Healthcare, Wauwatosa, U.S.A.) using linear (10 MHZ) and curvilinear (4 MHZ) probes. The length and echogenicity of the kidney as well as the corticomedullary definition were estimated. Furthermore, it was assessed whether the renal pelvic region was dilated. The blood flow in the kidney was evaluated using color Doppler.

### Necropsy

Four days after transplantation, the pigs were sacrificed in their home pens by an IV overdose of pentobarbital sodium (Euthasol vet. 400 mg/mL, Virbac).

All pigs, both donors and recipients, were examined post-mortem by a veterinary pathologist at the Department of Biomedical Sciences and Veterinary Public Health, Section of Pathology, SLU, Uppsala.

### Statistical Analyses

The statistical analyses were performed using Prism macOS Version 9.4.1 (458). One sample, unpaired and paired and multiple unpaired t-tests are used as indicated in the legends. Also, one-way ANOVA and 2-way repeated measures ANOVA followed by multiple comparisons testing without and with Bonferroni correction were employed. The results of the ANOVA are presented in the legends of the figures. The results of the t-tests and the multiple comparisons tests are presented as ns, * p<=0.05, ** p<0.01, *** p<0.001, **** and p<0.0001 in conjunction with the specific data plots. The specific statistical method is described in the legend of the figures.

### Results

### PEG-Phospholipid-Mediated Attenuation of Thromboinflammation Induced by Ischemia Reperfusion in Three Porcine Transplantation Models

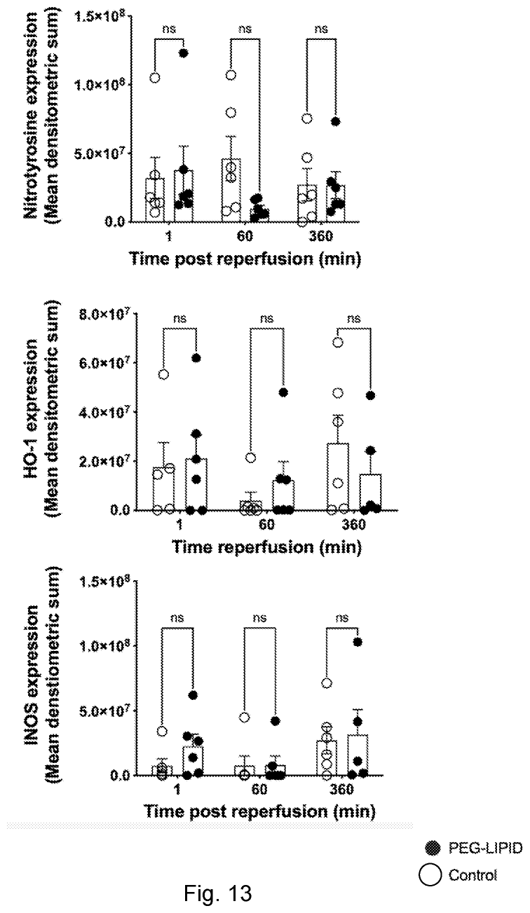

In order to evaluate the ischemic status of the PEG-phospholipid treated compared to the non-treated kidneys, biopsies were collected after 1, 60 and 360 min in the ANSM and stained for nitrotyrosine, HO-1 and INOS. No difference was found between the treated and non-treated kidneys, indicating that the tissue was in a similar ischemic condition (FIG. 13).

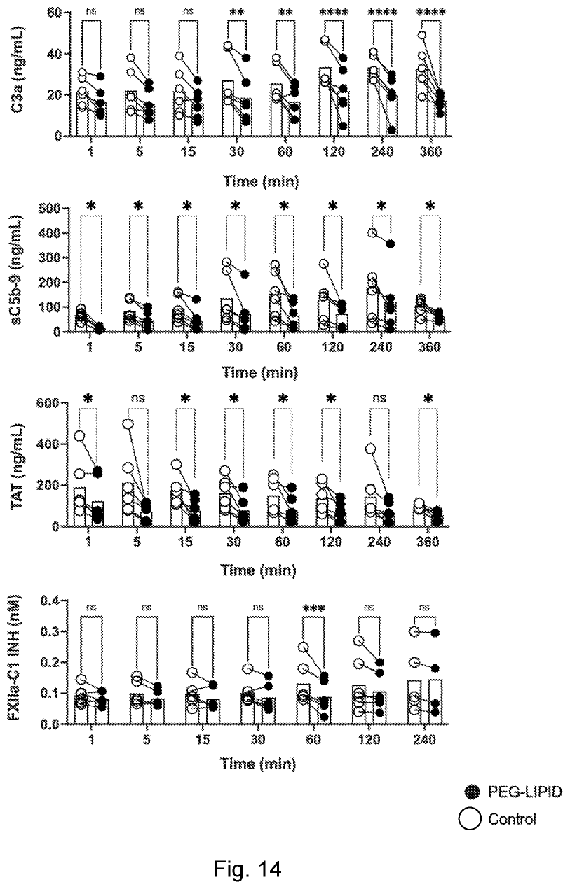

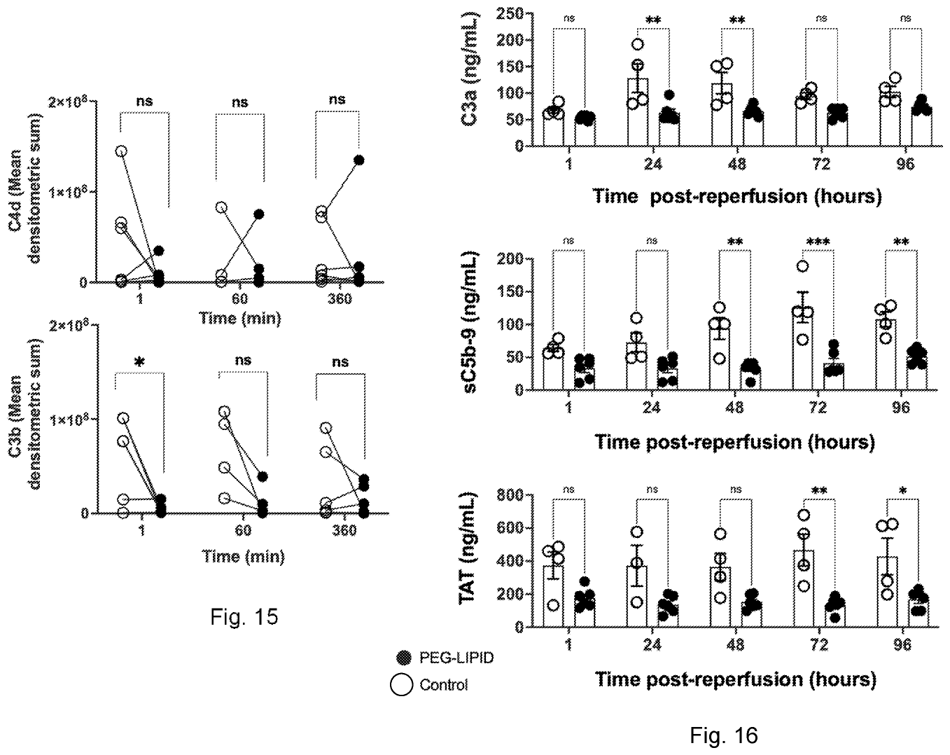

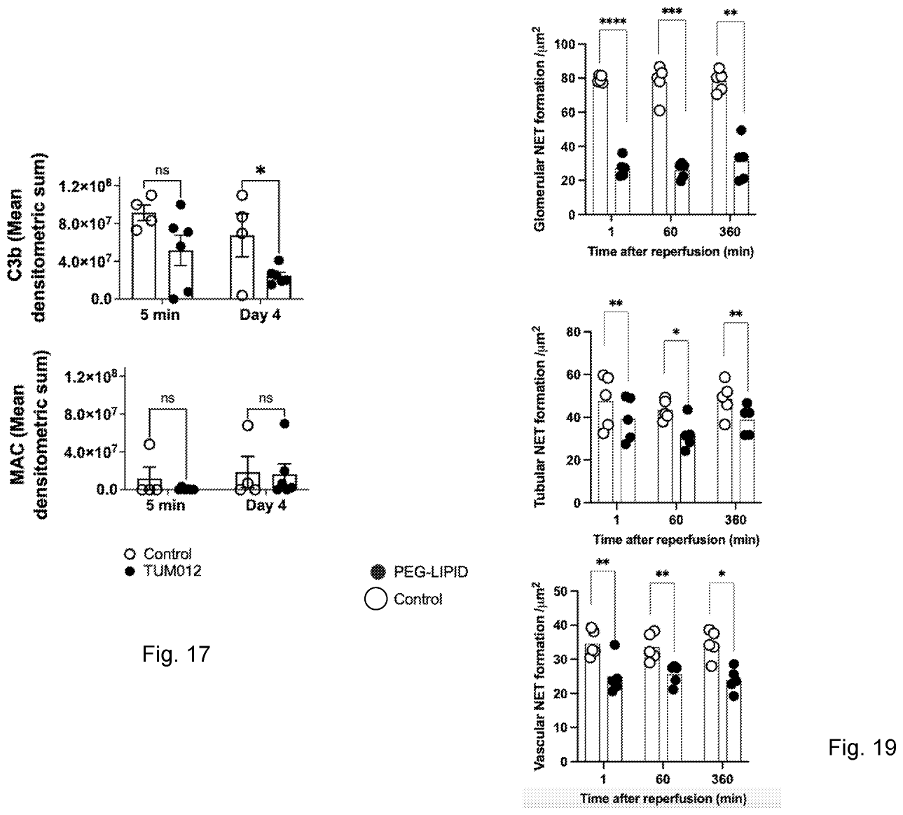

In the ANSM, biomarkers of thromboinflammation in blood samples collected from the renal vein of each kidney were analyzed. It was found that complement (C3a, sC5b-9), coagulation (TAT) and contact system (FXIla-C1INH) markers were substantially lower in the treated kidneys compared to the untreated, during the 6 h observation period (FIG. 14). Biopsies at 1 min, revealed local deposition of complement fragments C4d and C3b. There was a significantly lower binding of C3b in the PEG-phospholipid treated kidneys and a tendency of a lower binding of C4d in the treated kidneys (FIG. 15). This pattern was confirmed in the SM where the levels of C3a, sC5b-9 and TAT in the blood samples were substantially lower during the whole observation period (up to 4 days post-transplantation) in the PEG-phospholipid treated kidneys (FIG. 16). Renal biopsies taken at 5 min and at necropsy day 4 did also show a lower binding of C3b (2-way ANOVA) (FIG. 17). Although displaying lower values, the results from the CLSM confirmed these results (not shown).

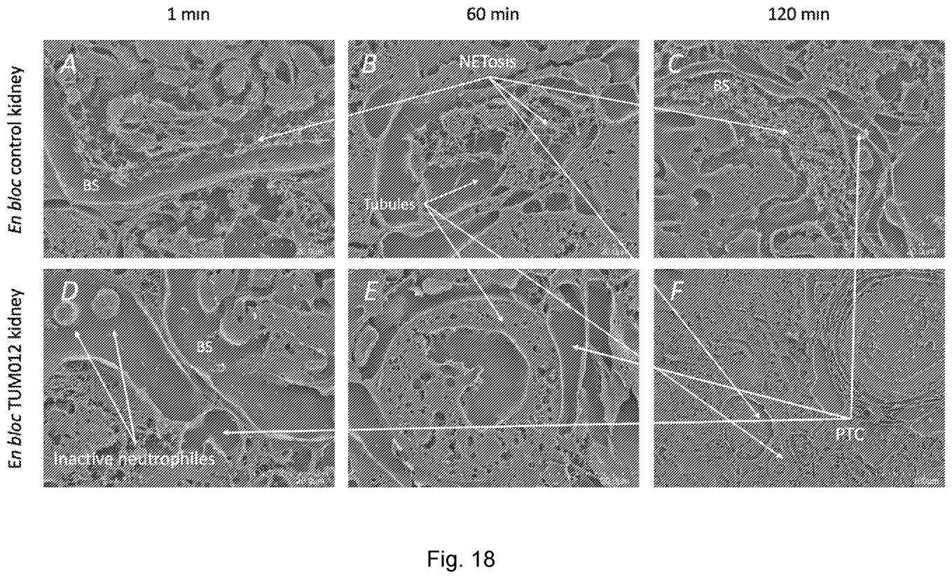

Furthermore, neutrophil extracellular traps (NETs) as one driving force of thromboinflammation, were analyzed in kidney graft biopsies from the ANSM by scanning electron microscopy (SEM; FIG. 18). NET depositions were found predominantly in the glomerular and tubular cavity already 1-minute post reperfusion. Quantification of renal NET deposition showed a marked decrease of both glomerular and tubular NETosis in PEG-phospholipid treated kidneys when compared to their matched individual controls (FIG. 19).

### PEG-Phospholipid-Mediated Attenuation of Systemic Inflammation Induced by Ischemia Reperfusion in Three Porcine Transplantation Models

In the ANSM, both the mRNA expression in kidney biopsies (RT-PCR, FIG. 20) and the plasma concentration in the renal vein (FIG. 21) of proinflammatory cytokines (IL-1β, IL-6, TNF) and TF were lower in the PEG-phospholipid treated animals compared to the corresponding controls. Particularly, IL-6 was clearly suppressed at most time points.

In the SM, the proinflammatory cytokines reflecting the local inflammation after 5 and 60 min and the systemic inflammation at 4 days post-transplantation were also assessed (FIG. 22). An inflammatory response was found, although not significant, already after 5 min, which was only observed in the untreated kidneys. After 60 min this inflammatory response was gone, and a steady state reached. Four days later a strong response engaging most of the analyzed cytokines, except for IL-8 and IL-1RA, was observed specifically in the pigs transplanted with untreated kidneys. Thus, when the sustained effects of ex vivo graft coating with PEG-phospholipid were studied, the attenuation of the systemic inflammation was clearly evident 4 days post transplantation. Considering the short duration of the PEG-phospholipid coating, this indicates that the PEG-phospholipid-mediated inhibition of the instantaneous thromboinflammation triggered by the IRI, also affects the long-lasting down-stream systemic inflammation. The results from the CLSM confirmed these results (not shown).

No toxicity or safety concerns regarding clinical, laboratory, or pathology parameters were observed in any of the transplantation models, which includes differences in general histology (hematoxylin staining) or macroscopic appearance between treated and non-treated kidneys.

### The Functional Effects of Ex Vivo PEG-Phospholipid Treatment in the Porcine Transplantation Models

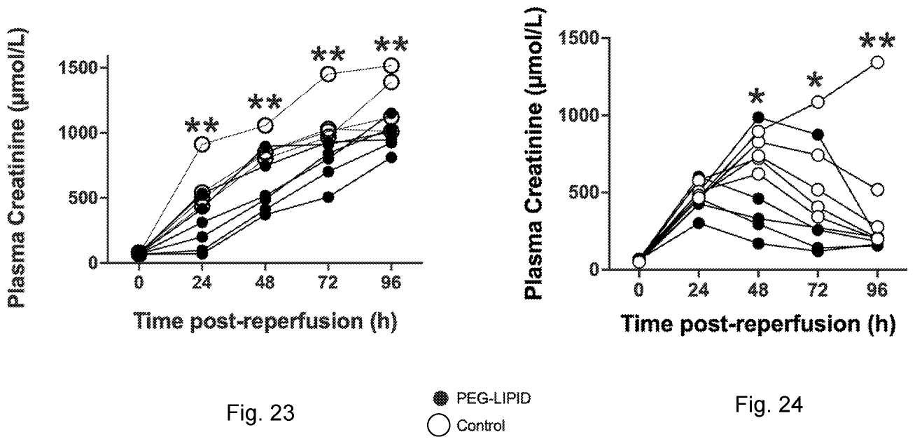

The kidney function was evaluated by diuresis post reperfusion in the ANSM and monitoring creatinine levels in collected blood samples from the recipients in the SM over time and found that creatinine levels were significantly lower post-transplantation in the pigs with grafts coated with PEG-phospholipid than in control grafts despite a very severe reperfusion injury with progressive kidney insufficiency (FIG. 23). Consistent with this finding was a similar effect of the coating in the CLSM, in which the IRI was less pronounced (FIG. 24).

The mechanism by which IRI is induced is not yet established, but binding of MBL, CL-11, MASP-2 and natural IgM antibodies have been suggested to bind to ligands on the ischemic cells thereby triggering the IRI. The PEG-phospholipid coating of the invention shields off the endothelial and tubular cell membranes by hindering cell membrane proteins to reach out and bind plasma proteins in the blood in the kidney graft after reperfusion. The data corroborates this concept in that the PEG-phospholipid effectively shields off small surface antigens, such as CD52 and the Rh antigens, which are extending approximately 1 nm from the surface (see Example 1). Also, CD8, which is ranging between 5-10 nm based on the crystal structure of the non-membranous part, is partially blocked by the PEG-phospholipid coat, while the PEG-phospholipid layer did not reach out to cover taller antigens such as CD4 with the estimated molecular extension of 15 nm from the membrane. Also, antibodies to CD3135 and vWF (extending 15 nm and 30 nm, respectively, from the surface) were not affected (see Example 3). These results are compatible with that PEG-phospholipid has a 5 kDa PEG chain with a theoretical 6 nm extension out from the cell membrane (FIG. 4).

To demonstrate the effect of the PEG-phospholipid on IRI, non-survival and survival porcine transplantation models were used, in which the recipients were non-immunosuppressed and followed for up to 4 days. In these in vivo transplantation models, the PEG-phospholipid treated kidneys compared to the non-treated ones, showed much less innate immune activation as reflected in attenuated plasma levels of TAT, FXIIa-C1INH, TF, C3a and sC5b-9, and of C3b deposition in the kidney biopsies. This demonstrated that the PEG-phospholipid construct could inhibit coagulation, contact and complement system activation and protect the cell membrane from C3b deposition after reperfusion of blood from the recipients, all of which are hallmarks of IRI. Corroborating these findings, the expression of proinflammatory cytokines such as IL-1β, IL-6, TNF and INFγ and of NETs formation were all attenuated in the PEG-phospholipid treated kidneys in the ANSM study, indicating a much lower thromboinflammatory reaction in the treated group. This pattern was much more pronounced after 4 days in the survival model where a very low expression of all tested cytokines was found in the treated group while in the non-treated group most cytokines were increased with the exception of IL-8 and IL-1RA. The profound effect demonstrated that the PEG-phospholipid provides an extensive protection against IRI and systemic inflammation after transplantation, which is conveyed into the adaptive immune response giving rise to humoral and cellular rejections. The PEG-phospholipid could also protect against the adaptive immune system and antibody-mediated rejection (AMR). The consistent pronounced inhibition of IL-6 in all three transplantation models supports this notion, since IL-6/IL-6R signaling inhibition is considered a novel therapeutic option for the prevention and treatment of allograft injury. Evidence from clinical trials supports the use of IL-6 blockade for desensitization and treatment of AMR in kidney transplantation (Curr Transplant Reports 8:1-14 (2021), J Am Soc Nephrol 20:1032-1040 (2009), Transplantation 101:32-44 (2017)).

### Example 6

The present Example assessed the ability of a PEG-phospholipid to block binding of anti-human leukocyte antigen (HLA) antibodies to freshly isolated human peripheral blood mononuclear cell (PBMC) expressing HLA antigens. HLA antigens are proteins on all nuclear cell surfaces, which present foreign peptides to the T-cell receptors on T cells in a cellular immune response. Antibodies against these structures are formed during allogeneic immune reactions, such as in blood transfusions and transplantations, and constitute the majority of all so-called donor specific antibodies (DSA). These antibodies are the main cause of antibody-mediated rejection (AMR). In order to avoid such incompatibility reactions a cross match is performed before transplantation, in which the patient's serum that may contain DSA, and PBMCs from the donor are mixed. If the DSA do not recognize the HLA antigens and no lysis of the donor PBMC cells occur, the cross match is considered to be negative, and the transplantation can be performed. If on the other hand the cross match is positive, the transplant organ has to be passed on to and matched (selected) with other patients with regard to HLA antigens. When a cross match combination is negative for donor and recipient, the transplantation can be performed.

### Materials and Methods

To test the efficacy of the PEG-phospholipid (SUNBRIGHT®) PP-050CN, N-(methylpolyoxyethylene oxycarbonyl)-1,2-dipalmitoyl-sn-glycero-3-phosphoethanolamine, sodium salt, NOF Corp., Japan) to block binding of anti-HLA antibodies to HLA antigens, similar to in a potential clinical setting, we used human PBMCs from healthy donors to block the HLA antigens.

### Materials

FITC Mouse Anti-Human HLA-ABC; Monoclonal (G46-2.6) (BD Pharmingen) cat no. 555552
Biotin Mouse Anti-human HLA Class I antibody [W6/32] (Abcam) cat no. ab110665
PE-streptavidin (BD Pharmingen) cat no. 554061

### Methods

PBMC was isolated from whole blood by using Cytiva™ Ficoll-Paque™ PLUS Media (Cytiva 17-1440-02) following standard protocol. In brief, whole blood was diluted in equal volume of PBS and carefully layered on top of Ficoll paque and centrifuged at 800×g for 25 mins. A cloudy layer was separated from the top of Ficoll and washed twice again with PBS.

The cells were stained with FITC mouse anti-human HLA-ABC monoclonal antibodies and biotin mouse anti-human HLA class | antibody [W6/32] (Abcam) followed by PE-streptavidin. The stained cells were analyzed by Beckman coulter cytoflex S (Biovis, facility, IGP, Rudbeck laboratory).

### Results

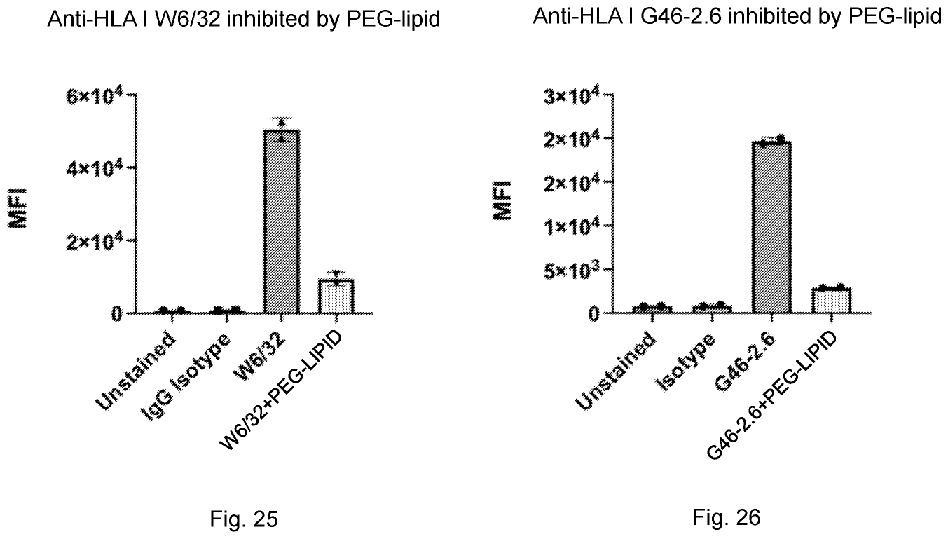

The results presented in FIGS. 25 and 26 show a robust inhibition of both anti-HLA antibodies to the PEG-phospholipid treated PBMCs indicating a strong masking effect mediated by the PEG-phospholipid. Accordingly, the PEG-phospholipid was capable of masking HLA antigens on the PBMCs and can thereby be used to block DSAs from binding to such HLA antigens on organ transplants, and thereby suppressing AMR.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-30. (canceled)

31. A method for inhibiting antibody-mediate rejection (AMR), the method comprises:
selective masking of surface antigens of an organ transplant comprising erythrocytes, thrombocytes and/or endothelial cells from a donor using poly(ethylene glycol) phospholipid (PEG-phospholipid) molecules to inhibit antibody binding to the surface antigens; and
transplanting the organ transplant into an uncrossmatched or incompatible recipient.

32. The method according to claim 31, wherein the organ transplant is selected from the group consisting of kidney, liver, pancreas, heart, lung, uterus, urinary bladder, thymus and intestine.

33. The method according to claim 31, wherein the surface antigens are human leukocyte antigens (HLAs) selected from the group consisting of HLA A, HLA B and HLA C.

34. The method according to claim 31, wherein the surface antigens are Rhesus factors selected from the group consisting of RhD, RhC, Rhc, RhE and Rhe.

35. The method according to claim 31, wherein the surface antigens are blood group antigens selected from the group consisting of A antigen and B antigen.

36. The method according to claim 31, wherein the surface antigens are human platelet antigens (HPAs) selected from the group consisting of HPA-1, HPA-2, HPA-3, HPA-4, HPA-5, HPA6, HPA-9 and HPA-15.

37. The method according to claim 31, the PEG-phospholipid molecules have an average extracellular length, when anchored in a cell membrane, selected within an interval of from 4 nm up to 8 nm.

38. The method according to claim 31, wherein the PEG-phospholipid molecules have a formula (I): 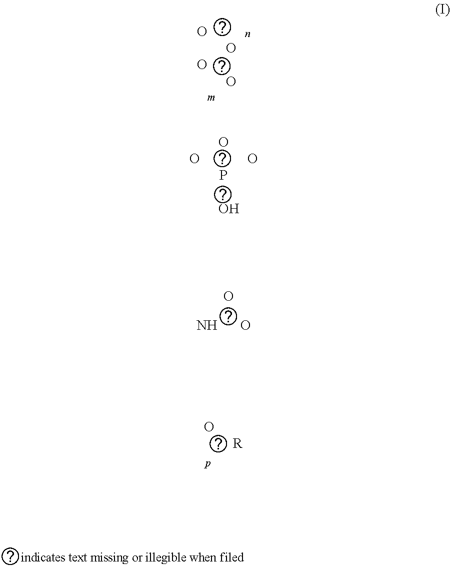
wherein
n, m are integers independently selected within the range of from 10 up to 16;
p is selected so that the PEG chain has an average molecular weight selected within the range of from 3 000 up to 10 000 Da; and
R is selected from the group consisting of H, methyl, and C1-C4 alkyl amine.

39. The method according to claim 31, wherein the PEG-phospholipid molecules have an average molecular weight as determined by gel permeation chromatography selected within an interval of from 5 to 7 kDa.

40. The method according to claim 31, wherein lipid part of the PEG-phospholipid molecules is 1,2-dipalmitoyl-sn-glycerol-3-phosphatidylethanolamine (DPPE).

41. The method according to claim 31, wherein the PEG part of the PEG-phospholipid molecules has an average degree of polymerization selected within an interval of from 105 to 125.

42. The method according to claim 31, wherein the PEG-phospholipid molecules are N-(methylpolyoxyethylene oxycarbonyl)-1,2-dipalmitoyl-sn-glycero-3-phosphoethanolamine.

43. The method according to claim 31, wherein the PEG-phospholipid molecules are not interconnected to other PEG-phospholipid molecules with any linker.

44. A blood product comprising erythrocytes and/or thrombocytes and poly(ethylene glycol) phospholipid (PEG-phospholipid) molecules anchored in the cell membrane of the erythrocytes and/or thrombocytes and masking surface antigens of the erythrocytes and/or thrombocytes, wherein a PEG chain of the PEG-phospholipid molecules has an average molecular weight selected within the range of from 3 000 up to 10 000 Da.

45. The blood product according to claim 44, wherein the surface antigens are Rhesus factors selected from the group consisting of RhD, RhC, Rhc, RhE and Rhe.

46. The blood product according to claim 44, wherein the surface antigens are blood group antigens selected from the group consisting of A antigen and B antigen.

47. The blood product according to claim 44, wherein the surface antigens are human platelet antigens (HPAs) selected from the group consisting of HPA-1, HPA-2, HPA-3, HPA-4, HPA-5, HPA6, HPA-9 and HPA-15.

48. The blood product according to claim 44, the PEG-phospholipid molecules have an average extracellular length, when anchored in cell membrane of erythrocytes, selected within an interval of from 4 nm up to 8 nm.

49. The blood product according to claim 44, wherein the PEG-phospholipid molecules have a formula (I): 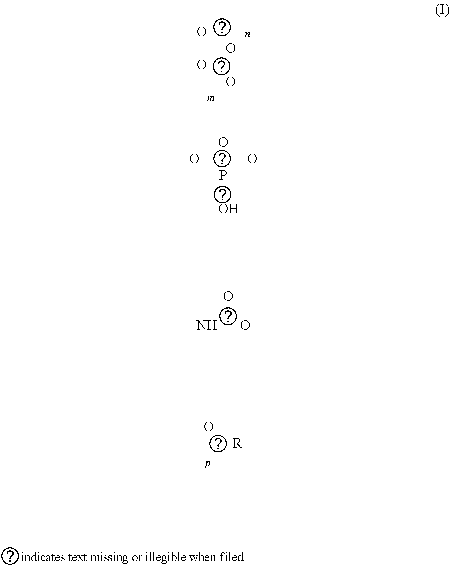
wherein
n, m are integers independently selected within the range of from 10 up to 16;
p is selected so that the PEG chain has an average molecular weight selected within the range of from 3 000 up to 10 000 Da; and
R is selected from the group consisting of H, methyl, and C1-C4 alkyl amine.

50. The blood product according to claim 44, wherein the PEG-phospholipid molecules have an average molecular weight as determined by gel permeation chromatography selected within an interval of from 5 to 7 kDa.

51. The blood product according to claim 44, wherein lipid part of the PEG-phospholipid molecules is 1,2-dipalmitoyl-sn-glycerol-3-phosphatidylethanolamine (DPPE).

52. The blood product according to claim 44, wherein the PEG part of the PEG-phospholipid molecules has an average degree of polymerization selected within an interval of from 105 to 125.

53. The blood product according to claim 44, wherein the PEG-phospholipid molecules are N-(methylpolyoxyethylene oxycarbonyl)-1,2-dipalmitoyl-sn-glycero-3-phosphoethanolamine.

54. The blood product according to claim 44, wherein the PEG-phospholipid molecules are not interconnected to other PEG-phospholipid molecules with any linker.

55. A blood transfusion method comprising:
adding poly(ethylene glycol)-lipid (PEG-phospholipid) molecules to a blood product from a donor to selectively mask surface antigens on erythrocytes and/or thrombocytes present in the blood product, wherein a PEG chain of the PEG-phospholipid molecules has an average molecular weight selected within the range of from 3 000 up to 10 000 Da; and
transfusing the blood product into an uncrossmatched or incompatible recipient.

56. The method according to claim 55, wherein the surface antigens are Rhesus factors selected from the group consisting of RhD, RhC, Rhc, RhE and Rhe.

57. The method according to claim 55, wherein the surface antigens are blood group antigens selected from the group consisting of A antigen and B antigen.

58. The method according to claim 55, wherein the surface antigens are human platelet antigens (HPAs) selected from the group consisting of HPA-1, HPA-2, HPA-3, HPA-4, HPA-5, HPA6, HPA-9 and HPA-15.

59. The method according to claim 55, the PEG-phospholipid molecules have an average extracellular length, when anchored in cell membrane of erythrocytes, selected within an interval of from 4 nm up to 8 nm.

60. The method according to claim 55, wherein the PEG-phospholipid molecules have a formula (I): 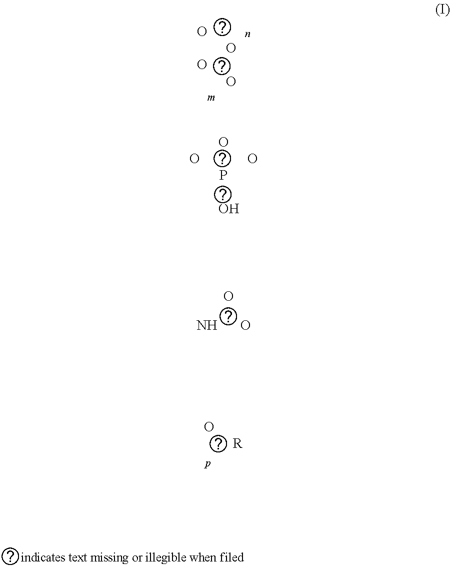
wherein
n, m are integers independently selected within the range of from 10 up to 16;
p is selected so that the PEG chain has an average molecular weight selected within the range of from 3 000 up to 10 000 Da; and
R is selected from the group consisting of H, methyl, and C1-C4 alkyl amine.

61. The method according to claim 55, wherein the PEG-phospholipid molecules have an average molecular weight as determined by gel permeation chromatography selected within an interval of from 5 to 7 kDa.

62. The method according to claim 55, wherein lipid part of the PEG-phospholipid molecules is 1,2-dipalmitoyl-sn-glycerol-3-phosphatidylethanolamine (DPPE).

63. The method according to claim 55, wherein the PEG part of the PEG-phospholipid molecules has an average degree of polymerization selected within an interval of from 105 to 125.

64. The method according to claim 55, wherein the PEG-phospholipid molecules are N-(methylpolyoxyethylene oxycarbonyl)-1,2-dipalmitoyl-sn-glycero-3-phosphoethanolamine.

65. The method according to claim 55, wherein the PEG-phospholipid molecules are not interconnected to other PEG-phospholipid molecules with any linker.

66. An in vitro method of treating erythrocytes and/or thrombocytes comprising selective masking surface antigens of erythrocytes and/or thrombocytes by adding in vitro poly(ethylene glycol)-lipid (PEG-phospholipid) molecules to the erythrocytes and/or thrombocytes, wherein a PEG chain of the PEG-phospholipid molecules has an average molecular weight selected within the range of from 3 000 up to 10 000 Da.

67. An irregular antibody screening method comprising:
adding poly(ethylene glycol)-lipid (PEG-phospholipid) molecules to a blood sample from a subject to selectively mask surface antigens on erythrocytes present in the blood sample, wherein a PEG chain of the PEG-phospholipid molecules has an average molecular weight selected within the range of from 3 000 up to 10 000 Da; and
screening for irregular antibodies bound to erythrocytes present in or obtained from the blood sample.
